(19) United States
(12) Patent Application Publication
(10) Pub. No.: US 2015/0037825 A1
(43) Pub. Date: Feb. 5, 2015

(54) Means and Methods for Assessing Hematopoietic Toxicity

(71) Applicant: BASF SE, Ludwigshafen (DE)

(72) Inventors: Tilmann B. Walk, Kleinmachnow (DE); Bennard van Ravenzwaay, Altrip (DE); Werner Mellert, Hassloch (DE); Eric Fabian, Speyer (DE); Volker Strauss, Bad Durkheim (DE); Hennicke Kamp, Bischheim (DE); Jan C. Wiemer, Berlin (DE); Ralf Looser, Berlin (DE); Michael Manfred Herold, Berlin (DE); Alexandre Prokoudine, Berlin (DE)

(73) Assignee: BASF SE, Ludwigshafen (DE)

(21) Appl. No.: 14/344,517

(22) PCT Filed: Sep. 12, 2012
(86) PCT No.: PCT/IB2012/054731

§ 371 Date: Oct. 8, 2014

Related U.S. Application Data
(60) Provisional application No. 61/533,867, filed on Sep. 13, 2011.

(30) Foreign Application Priority Data
Sep. 13, 2011 (EP) 11181156.8

(51) Int. Cl. G01N 33/53

(52) U.S. Cl. CPC G01N 33/53; G01N 2500/04; G01N 2800/709
USPC 435/7.94; 435/7.92; 435/287.2

(57) ABSTRACT

The present invention pertains to the field of diagnostics for hematopoietic toxicity and toxicological assessments for risk stratification of chemical compounds. Specifically, it relates to a method for diagnosing hematopoietic toxicity. It also relates to a method for determining whether a compound is capable of inducing such hematopoietic toxicity in a subject and to a method of identifying a drug for treating hematopoietic toxicity. Furthermore, the present invention relates to a device and a kit for diagnosing hematopoietic toxicity.

The present invention pertains to the field of diagnostics for hematopoietic toxicity and toxicological assessments for risk stratification of chemical compounds. Specifically, it relates to a method for diagnosing hematopoietic toxicity. It also relates to a method for determining whether a compound is capable of inducing such hematopoietic toxicity in a subject and to a method of identifying a drug for treating hematopoietic toxicity. Furthermore, the present invention relates to a device and a kit for diagnosing hematopoietic toxicity.

Bone marrow is one of the largest organs in the body and is an important potential target organ of chemical exposure. The bone marrow is found within the central cavities of axial and long bones. It consists of hematopoietic tissue islands and adipose cells surrounded by vascular sinuses interspersed within a meshwork of trabecular bone. The bone marrow is the major hematopoietic organ, and a primary lymphoid tissue, responsible for the production of erythrocytes, granulocytes, monocytes, lymphocytes and platelets. The inner surface of the bone cavities and the outer surface of the cancellous bone spicules within the cavities are covered by an endosteal lining consisting of a single layer of flat “bone-lining cells” supported by a thin layer of reticular connective tissue; osteoblasts and osteoclasts are also found within the endosteal lining. In long bones, one or more nutrient canals pass through the cortical bone entering the marrow cavity obliquely. In flat bones, the marrow is served by numerous blood vessels of various sizes entering the marrow via large and small nutrient canals.

The marrow has an extensive blood supply. Also, it appears that nutrient artery-derived capillaries extend into the Haversian canals, return to the marrow cavity then open into the venous sinuses. Thus, there is a circular pattern to blood flow within the marrow cavity, from the center of the marrow cavity toward the periphery of the marrow cavity then back toward the center. In long and flat bones, the blood supplies of the bone and bone marrow are interconnected through an endosteal network of vessels.

Bone marrow innervation occurs with myelinated and nonmyelinated nerves that enter through the nutrient canals. Some innervation also occurs through epiphyseal and metaphyseal foramina. Nerve bundles follow the arterioles with branches serving the smooth muscle of the vessels or, occasionally, terminating in the hematopoietic tissue amongst hematopoietic cells.

The hematopoietic tissue consists of a variety of cell types including, the blood cells and their precursors, adventitial/barrier cells, adipocytes, and macrophages. The hematopoietic tissue cells are not randomly arranged but demonstrate a particular organization within the tissue. For hematopoiesis it must be supported by a microenvironment that is able to recognize and retain hematopoietic stem cells and provide the factors required to support proliferation, differentiation and maturation of stem cells along committed lineages.

Hematopoiesis is a compartmentalized process within the hematopoietic tissue with erythropoiesis taking place in erythroblastic islands; granulopoiesis occurs in less distinct foci and megakaryopoiesis occurs adjacent to the sinus endothelium. Upon maturation, the hematopoietic cells, regulated by the barrier cells, traverse the wall of the venous sinuses to enter the bloodstream; platelets are released directly into the blood from cytoplasmic processes of megakaryocytes penetrating through the sinus wall into the sinus lumen.

The production, differentiation, and maturation of blood cells are regulated by humoral factors. Some factors act on the more primitive cells and have a general action, while others (e.g., erythropoietin) act on later progenitors of a specific cell line. The sources of hematopoietic factors vary.

Hematopoiesis is a continuous process, but can be separated into distinct stages. The first stage involves uncommitted (pluripotent) stem cells contained in the bone marrow. These pluripotent cells have two primary functions. First, they maintain their numbers by a process of self renewal and, secondly, they have the ability to give rise to all hematopoietic cells. They also appear to be found in greater numbers peripherally from the central axis, near the bone lining cells.

Lymphopoiesis occurs within the bone marrow microenvironment of adult mammals. B-lineage cells derived from the marrow can be identified by sequential changes in cell size and expression of immunoglobulin chains. The sequence of proliferation/maturation of B-lymphopoiesis is regulated by soluble and is sensitive to disruption by myelotoxic chemicals. For example, polyhydroxy metabolites of benzene (e.g., hydroquinone) have been shown to affect B-lymphopoiesis

Exposure to a variety of drugs and toxins induces bone marrow injury and alters hematopoiesis. Since the marrow has a large reserve capacity, only widespread and severe marrow damage results in alterations in cell counts in the peripheral blood. The pathogenesis of bone marrow injury remains obscure for most toxic agents. Although some agents, most notably chemotherapeutic agents, induce predictable, dose-dependent toxicity of rapidly proliferating marrow precursors, many agents produce idiosyncratic marrow damage. Direct bone marrow damage may interfere with the marrow's ability to mount appropriate systemic responses. Alternatively, marrow damage may be reflected by maturation abnormalities in any or all of the proliferating marrow cell lines. This in turn can cause a variety of peripheral blood aberrations as well as morphologic abnormalities in the marrow. On the other hand, when the marrow is the primary effector organ, proliferative responses in one or more cell lines may reflect an appropriate direct compound-related effect rather than a compensatory response to a systemic problem.

Interpretation of marrow changes in a toxicological setting may be quite complex and may involve both local as well as systemic manifestations of toxicity and/or pharmacologic response. In a general way, marrow changes can be classified as either quantitative or qualitative. Quantitative abnormalities include the various hyperplasias and hypoplasias of the proliferating cell lines and require simultaneous evaluation of peripheral blood data for proper interpretation. Qualitative abnormalities refer to morphologic aberrations in marrow precursors (marrow dysplasias) as well as changes such as marrow necrosis, macrophage hyperplasia, and plasmacytosis.

Bone marrow toxicity is a special type of maturation arrest in which both cytoplasm and nucleus may be affected. Systemic toxemia may affect the development of cells of all proliferating cell lines; however, toxicity is most easily recognized in late-stage granulocyte precursors (metamyelocytes, band cells, and mature neutrophils). Bone marrow toxicity may be drug-induced, associated with circulating bacterial toxins in cases of severe infection, or caused by the circulating toxins released from sites of extensive tissue necrosis.

Due to the diversity of possible actions, the assessment of bone marrow toxicity with regards to suppression and mineralization is a rather complex process. The current methods usually comprise hematological investigations, pathological and histopathological investigations as well as a biochemical analysis. However, the biomarkers are rather complex regulated and changes may sometimes occur even at rather progressed stages. Major drawbacks of the histopathological assessments are that they are invasive, and even when combined with the clinical pathology/hematology measurements that they are less reliable because they are in part based an the individual interpretations of toxicologist carrying out the investigations. (see, e.g., Andrews C M (1998) The haematopoieticy system, in: Target organ pathology, a basic text, Turton J and Hooson J (eds) Taylor & Francis, London, United Kingdom, 1998; Heaney R P, Whedon G D (2010) Bone morphology, in: Encyclopedia Britannica. Retrieved Oct. 26, 2010, from Encyclopedia Britannica Online: http://www.britannica.com/EBchecked/topic/72869/bone/41883/Bone-morphology; Re-bar 1993, Toxicol. Pathol. 21: 118-129; Travlos 2006, Toxicol. Pathol. 35: 548-565; Weiss 1993, Toxicol. Pathol. 21: 135-140).

Sensitive and specific methods for determining efficiently and reliably bone marrow toxicity and, in particular, the early onset thereof are not available but would, nevertheless, be highly appreciated. The importance of bone marrow toxicity may become apparent if one considers its consequences on hematopoiesis including lymphopoiesis. Moreover, chemical compounds which are used in any kind of industry in the European Community, e.g., will now need to comply with REACH (Registration, Evaluation and Authorisation of Chemicals). it will be understood that the potential of a chemical compound to induce bone marrow toxicity with regards to suppression and mineralization will be deemed as a high risk for the compound and, consequently, the compound will be available only for limited applications and when obeying high security standards.

Another important hematopoietic organ which may become affected by hematopoietic toxicity is blood. Blood is one of the largest organs in the body and is an important potential target organ of chemical exposure. Blood is a rapidly dividing tissue and blood-forming capacity has a high potential for expansion. In human the turnover of blood cells is considerable, of the order of 2 to 3×10¹¹ cells per day. The bone marrow is the major hematopoietic organ responsible for the production of erythrocytes, granulocytes, monocytes, lymphocytes and platelets. Hematopoiesis is a compartmentalized process within the hematopoietic tissue with erythropoiesis taking place in erythroblastic islands; granulopoiesis occurs in less distinct foci and megakaryopoiesis occurs adjacent to the sinus endothelium. Upon maturation, the hematopoietic cells traverse the wall of the venous sinuses to enter the bloodstream; platelets are released directly into the blood from cytoplasmic processes of megakaryocytes penetrating through the sinus wall into the sinus lumen. The production, differentiation, and maturation of blood cells are regulated by humoral factors. With the exception of erythrocytes, the other cell types are on their way to a location where their function is required. All the cell types are constantly leaving the circulation and being replaced at differing rates.

Erythrocytes make up 40 to 45 percent of the circulating blood volume and serve as the principal vehicle of transportation of oxygen from the lungs to the peripheral tissues. In addition, erythrocytes are involved in the transport of carbon dioxide from tissues to the lung and in the maintenance of a constant pH in blood. Erythrocytes help modulate the inflammatory response and/or are a reservoir for drugs and toxins. Erythrocyte production is a continuous process that is dependent on frequent cell division and a high rate of hemoglobin synthesis. Synthesis of hemoglobin is dependent on coordinated production of globin chains and heme moieties. Synthesis of heme requires incorporation of iron into a porphyrin ring. Iron deficiency is usually the result of dietary deficiency or increased blood loss. Any drug that contributes to blood loss, such as nonsteroidal anti-inflammatory agents, with their increased risk of gastrointestinal ulceration and bleeding, may potentiate the risk of developing iron deficiency anemia. Defects in the synthesis of porphyrin ring of heme can lead to sideroblastic anemia, with its characteristic accumulation of iron in bone marrow erythroblasts.

Drug-induced aplastic anemia may represent either a predictable or idiosyncratic reaction to a xenobiotic. This life-threatening disorder is characterized by peripheral blood pancytopenia, reticulocytopenia, and bone marrow hypoplasia. Agents such as benzene and radiation have a predictable effect on hematopoietic progenitors, and the resulting aplastic anemia corresponds to the magnitude of the exposure to these agents. In contrast, idiosyncratic aplastic anemia does not appear to be related to the dose of the agent initiating the process. There are many agents has been associated with the development of aplastic anemia, many of which have been reported in only a few patients. Aplastic, or non-regenerative, anemia is a syndrome associated with bone marrow failure, characterized by anemia, pancytopenia, and varying degrees of bone marrow hypocellularity.

Aplastic anemia is classified as idiopathic or secondary, depending on whether its onset can be attributed to known causes, for example, ionizing radiation, drug, or chemical exposure. Aplastic anemia is a disorder of stem cell regulation, either through exhaustion of numbers, or a defect in differentiation, are unable to recapitulate blood cells. Stromal cell defects may also play an important role in chronic bone marrow failure. In some of these cases there is evidence to support a clonal origin for aplastic anemia. Animal models of aplastic anemia are relatively few, and have been largely restricted to those induced by viruses, busulfan, irradiation, or benzene. The bone marrow has long been recognized as particularly susceptible to radiation-induced aplastic anemia in many species, including dogs, monkeys and mice. Aplastic anemia is also sometimes associated with exposure to drugs, including chloramphenicol, carbamazepine, felbamate, phenytoin, quinine, and phenylbutazone.

Lead has multiple hematologic effects among others, it decreases the ferrochelatase activity. This enzyme catalyzes the incorporation of the ferrous ion into the porphyrin ring structure. Failure to insert iron into protoporphyrin results in depressed heme formation. The excess protoporphyrin takes the place of heme in the hemoglobin molecule and, as the red blood cells containing protoporphyrin circulate, zinc is chelated at the center of the molecule at the site usually occupied by iron. Red blood cells containing zinc-protoporphyrin are intensely fluorescent and may be used to diagnose lead toxicity. Depressed heme synthesis is thought to be the stimulus for increasing the rate of activity of the first step in the heme synthetic pathway.

Hemostasis is a multicomponent system responsible for preventing the loss of blood from sites of vascular injury and maintaining circulating blood in a fluid state. The major constituents of the hemostatic system include circulating platelets, a variety of plasma proteins, and vascular endothelial cells. Platelets are essential for formation of a stable hemostatic plug in response to vascular injury. They are formed from the mature megakaryocyte, a polyploid cell. The mechanism by which platelets are released is unclear, but appears to be by fragmentation of the cytoplasm. The cytokine, thrombopoietin, stimulates megakaryocyte proliferation, platelet production, and differentiation from the common stem cell. The lifespan of platelets varies from species to species: in human it is 10 days, in the dog 8 days, in the rat 4.5 days and in the mouse 4 days. Similarly, mean platelet volume is lower (and platelet count greater) in rodents than in human; in dogs and cats platelet volume is higher than in man. Platelets initially adhere to the damaged wall through binding of von Willebrand factor (vWF) with the platelet glycoprotein Ib/IX/V (GP Ib/IX/V) receptor complex.

Xenobiotics may interfere with the platelet response by causing thrombocytopenia or interfering with platelet function; some agents are capable of affecting both platelet number and function. Platelet function is dependent on the coordinated interaction of a number of biochemical response pathways. A variety of drugs and foods have been found to inhibit platelet function. Major drug groups that affect platelet function include nonsteroidal anti-inflammatory agents, beta-lactam-containing antibiotics, cardiovascular drugs, particularly beta blockers, psychotropic drugs, anesthetics, antihistamines, and some chemotherapeutic agents. Coagulation is the result of sequential activation of a series of serine proteases that culminates in the formation of thrombin. Thrombin is a multifunctional enzyme that converts fibrinogen to fibrin; activates factors V, VIII, XI, XIII, protein C, and platelets; and interacts with a variety of cells. The most common toxic effects of xenobiotics on fibrin clot formation are related to a decreased level of one or more of the critical proteins necessary for this process. The decrease in clotting factor activity may be due to decreased synthesis of the protein(s) or increased clearance from the circulation. The majority of proteins involved in the coagulation cascade are synthesized in the liver. Therefore, any agent that impairs liver function may cause a decrease in production of coagulation factors.

Exposure to a variety of drugs and toxins induces hematotoxicity characterized by aplastic anemia, inhibition of platelet aggregation and inhibition of porphyrin synthesis among others. Due to the diversity of possible actions, the assessment of hematotoxicity is a rather complex process. The current methods usually comprise hematological investigations, pathological and histopathological investigations as well as a biochemical analysis. However, the biomarkers are rather complex regulated and changes may sometimes occur even at rather progressed stages. Major drawbacks of the histopathological assessments are that they are invasive, and even when combined with the clinical pathology/hematology measurements that they are less reliable because they are in part based an the individual interpretations of toxicologist carrying out the investigations. (see, e.g., Aksoy 1989, Environ. Health Perspect. 82: 193-197; Andrews C M (1998) The haematopoieticy system, in: Target organ pathology, a basic text, Turton J and Hooson J (eds) Taylor & Francis, London, United Kingdom; 1998; Bloom J C, Brandt J T (2008) Chapter 11, Toxic responses of the blood, in: Casarett & Doull's Toxicology, The basic science of poisons, Klaassen C D (ed.), McGraw-Hill P, 7th revised edition, New York (2008); Haschek W M, Wallig M A, Rousseaux (2010) Fundamentals of toxicologic pathology, 2nd edition, Academic Press, Elsevier, London, UK).

Sensitive and specific methods for determining efficiently and reliably blood toxicity with regards to aplastic anemia, inhibition of platelet aggregation and porphyrine synthesis and, in particular, the early onset thereof are not available but would, nevertheless, be highly appreciated. The importance of blood toxicity may become apparent if one considers its consequences like aplastic anemia, inhibition of platelet aggregation and porphyrine synthesis. Moreover, chemical compounds which are used in any kind of industry in the European Community, e.g., will now need to comply with REACH (Registration, Evaluation and Authorisation of Chemicals). It will be understood that the potential of a chemical compound to induce blood toxicity, especially aplastic anemia, inhibition of platelet aggregation or porphyrine synthesis will be deemed as a high risk for the compound and, consequently, the compound will be available only for limited applications and when obeying high security standards.

Sensitive and specific methods for assessing the toxicological properties of a chemical compound and, in particular, hematopoietic toxicity, in an efficient and reliable manner are not yet available but would, nevertheless, be highly appreciated.

Thus, the technical problem underlying the present invention could be seen as the provision of means and methods for complying with the aforementioned needs. The technical problem is solved by the embodiments characterized in the claims and described herein below.

Accordingly, the present invention relates to a method for diagnosing hematopoietic toxicity comprising:
(a) determining the amount of at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, or 9 in a test sample of a subject suspected to suffer from hematopoietic toxicity, and
(b) comparing the amounts determined in step (a) to a reference, whereby hematopoietic toxicity is to be diagnosed.

In a preferred embodiment of the aforementioned method said subject has been brought into contact with a compound suspected to be capable of inducing hematopoietic toxicity.

The present invention also relates to a method of determining whether a compound is capable of inducing hematopoietic toxicity in a subject comprising:
(a) determining in a sample of a subject which has been brought into contact with a compound suspected to be capable of inducing hematopoietic toxicity the amount of at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, or 9; and
(b) comparing the amounts determined in step (a) to a reference, whereby the capability of the compound to induce hematopoietic toxicity is determined.

In a preferred embodiment of the aforementioned method said compound is at least one compound selected from the group consisting of: 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Saflufenacil, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen.

In another preferred embodiment of the methods of the present invention said reference is derived from (i) a subject or group of subjects which suffers from hematopoietic toxicity or (ii) a subject or group of subjects which has been brought into contact with at least one compound selected from the group consisting of: 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Saflufenacil, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen. In a more preferred embodiment of said method essentially identical amounts for the biomarkers in the test sample and the reference are indicative for hematopoietic toxicity.

In another preferred embodiment of the methods of the present invention said reference is derived from (i) a subject or group of subjects known to not suffer from hematopoietic toxicity or (ii) a subject or group of subjects which has not been brought into contact with at least one compound selected from the group consisting of: 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Saflufenacil, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen. In a more preferred embodiment of said methods amounts for the biomarkers which differ in the test sample in comparison to the reference are indicative for hematopoietic toxicity.

In yet another embodiment of the methods of the present invention said reference is a calculated reference for the biomarkers for a population of subjects. In a more preferred embodiment of said methods amounts for the biomarkers which differ in the test sample in comparison to the reference are indicative for hematopoietic toxicity.

The present invention also contemplates a method of identifying a substance for treating hematopoietic toxicity comprising the steps of:
(a) determining in a sample of a subject suffering from hematopoietic toxicity which has been brought into contact with a candidate substance suspected to be capable of treating hematopoietic toxicity the amount of at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9, 12 a or 12b; and
(b) comparing the amounts determined in step (a) to a reference, whereby a substance capable of treating hematopoietic toxicity is to be identified.

In a preferred embodiment of the aforementioned method said reference is derived from (i) a subject or group of subjects which suffers from hematopoietic toxicity or (ii) a subject or group of subjects which has been brought into contact with at least one compound selected from the group consisting of: 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Saflufenacil, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen. In a more preferred embodiment of said method amounts for the biomarkers which differ in the test sample and the reference are indicative for a substance capable of treating hematopoietic toxicity.

In another preferred embodiment of the aforementioned method said reference is derived from (i) a subject or group of subjects known to not suffer from hematopoietic toxicity or (ii) a subject or group of subjects which has not been brought into contact with at least one compound selected from the group consisting of: 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Saflufenacil, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen. In a more preferred embodiment of the said methods essentially identical amounts for the biomarkers in the test sample and the reference are indicative for a substance capable of treating hematopoietic toxicity.

In yet another preferred embodiment of the aforementioned method said reference is a calculated reference for the biomarkers in a population of subjects. In a more preferred embodiment of the said methods essentially identical amounts for the biomarkers in the test sample and the reference are indicative for a substance capable of treating hematopoietic toxicity.

The present invention also relates to the use of at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9, 12 a or 12b or a detection agent for the said biomarker for diagnosing hematopoietic toxicity in a sample of a subject.

Moreover, the present invention relates to a device for diagnosing hematopoietic toxicity in a sample of a subject suspected to suffer therefrom comprising:
(a) an analyzing unit comprising a detection agent for at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9, 12 a or 12b which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby hematopoietic toxicity is diagnosed.

In a preferred embodiment of the device of the invention said stored reference is a reference derived from a subject or a group of subjects known to suffer from hematopoietic toxicity or a subject or group of subjects which has been brought into contact with at least one compound selected from the group consisting of 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Saflufenacil, Cyclosporin A, Epoxiconazole, Flutamide, Lead acetate, trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen, and said data processor executes instructions for comparing the amount of the at least one biomarker determined by the analyzing unit to the stored reference, wherein an essentially identical amount of the at least one biomarker in the test sample in comparison to the reference is indicative for the presence of hematopoietic toxicity or wherein an amount of the at least one biomarker in the test sample which differs in comparison to the reference is indicative for the absence of hematopoietic toxicity.

In another preferred embodiment of the device of the invention said stored reference is a reference derived from a subject or a group of subjects known to not suffer from hematopoietic toxicity or a subject or group of subjects which has not been brought into contact with at least one compound selected from the group consisting of 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Saflufenacil, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen, and said data processor executes instructions for comparing the amount of the at least one biomarker determined by the analyzing unit to the stored reference, wherein an amount of the at least one biomarker in the test sample which differs in comparison to the reference is indicative for the presence of hematopoietic toxicity or wherein an essential identical amount of the at least one biomarker in the test sample in comparison to the reference is indicative for the absence of hematopoietic toxicity.

Further, the present invention relates to a kit for diagnosing hematopoietic toxicity comprising a detection agent for the at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9, 12a or 12b and standards for the at least one biomarker the concentration of which is derived from a subject or a group of subjects known to suffer from hematopoietic toxicity or derived from a subject or a group of subjects known to not suffer from hematopoietic toxicity.

In particular, the present invention relates to a method for diagnosing bone marrow toxicity comprising:
(a) determining the amount of at least one biomarker selected from any one of Tables 1a, 1b, 1c, 1d, 1e, or 1f in a test sample of a subject suspected to suffer from bone marrow toxicity, and
(b) comparing the amounts determined in step (a) to a reference, whereby bone marrow toxicity is to be diagnosed.

In a preferred embodiment of the aforementioned method said subject has been brought into contact with a compound suspected to be capable of inducing bone marrow toxicity.

The present invention also relates to a method of determining whether a compound is capable of inducing bone marrow toxicity in a subject comprising:
(a) determining in a sample of a subject which has been brought into contact with a compound suspected to be capable of inducing bone marrow toxicity the amount of at least one biomarker selected from any one of Tables 1a, 1b, 1c, 1d, 1e, or 1f; and
(b) comparing the amounts determined in step (a) to a reference, whereby the capability of the compound to induce bone marrow toxicity is determined.

In a preferred embodiment of the aforementioned method said compound is at least one compound selected from the group consisting of: Adriamycin hydrochloride, Carboplatin, Cisplatin, Cyclophosphamide monohydrate, Cytarabin, Ibuprofen, and Oxaliplatin.

In another preferred embodiment of the methods of the present invention said reference is derived from (i) a subject or group of subjects which suffers from bone marrow toxicity or (ii) a subject or group of subjects which has been brought into contact with at least one compound selected from the group consisting of: Adriamycin hydrochloride, Carboplatin, Cisplatin, Cyclophosphamide monohydrate, Cytarabin, Ibuprofen, and Oxaliplatin. In a more preferred embodiment of said method essentially identical amounts for the biomarkers in the test sample and the reference are indicative for bone marrow toxicity.

In another preferred embodiment of the methods of the present invention said reference is derived from (i) a subject or group of subjects known to not suffer from bone marrow toxicity or (ii) a subject or group of subjects which has not been brought into contact with at least one compound selected from the group consisting of: Adriamycin hydrochloride, Carboplatin, Cisplatin, Cyclophosphamide monohydrate, Cytarabin, Ibuprofen, and Oxaliplatin. In a more preferred embodiment of said methods amounts for the biomarkers which differ in the test sample in comparison to the reference are indicative for bone marrow toxicity.

In yet another embodiment of the methods of the present invention said reference is a calculated reference for the biomarkers for a population of subjects. In a more preferred embodiment of said methods amounts for the biomarkers which differ in the test sample in comparison to the reference are indicative for bone marrow toxicity.

The present invention also contemplates a method of identifying a substance for treating bone marrow toxicity comprising the steps of:
(a) determining in a sample of a subject suffering from bone marrow toxicity which has been brought into contact with a candidate substance suspected to be capable of treating bone marrow toxicity the amount of at least one biomarker selected from any one of Tables 1a, 1b, 1c, 1d, 1e, or 1f; and
(b) comparing the amounts determined in step (a) to a reference; whereby a substance capable of treating bone marrow toxicity is to be identified.

In a preferred embodiment of the aforementioned method said reference is derived from (i) a subject or group of subjects which suffers from bone marrow toxicity or (ii) a subject or group of subjects which has been brought into contact with at least one compound selected from the group consisting of: Adriamycin hydrochloride, Carboplatin, Cisplatin, Cyclophosphamide monohydrate, Cytarabin, Ibuprofen, and Oxaliplatin. In a more preferred embodiment of said method amounts for the biomarkers which differ in the test sample and the reference are indicative for a substance capable of treating bone marrow toxicity.

In another preferred embodiment of the aforementioned method said reference is derived from (i) a subject or group of subjects known to not suffer from bone marrow toxicity or (ii) a subject or group of subjects which has not been brought into contact with at least one compound selected from the group consisting of: Adriamycin hydrochloride, Carboplatin, Cisplatin, Cyclophosphamide monohydrate, Cytarabin, Ibuprofen, and Oxaliplatin. In a more preferred embodiment of the said methods essentially identical amounts for the biomarkers in the test sample and the reference are indicative for a substance capable of treating bone marrow toxicity.

In yet another preferred embodiment of the aforementioned method said reference is a calculated reference for the biomarkers in a population of subjects. In a more preferred embodiment of the said methods essentially identical amounts for the biomarkers in the test sample and the reference are indicative for a substance capable of treating bone marrow toxicity.

The present invention also relates to the use of at least one biomarker selected from any one of Tables 1a, 1b, 1c, 1d, 1e, or 1f or a detection agent for the said biomarker for diagnosing bone marrow toxicity in a sample of a subject.

Moreover, the present invention relates to a device for diagnosing bone marrow toxicity in a sample of a subject suspected to suffer therefrom comprising:
(a) an analyzing unit comprising a detection agent for at least one biomarker selected from any one of Tables 1a, 1b, 1c, 1d, 1e, or 1f which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby bone marrow toxicity is diagnosed.

In a preferred embodiment of the device of the invention said stored reference is a reference derived from a subject or a group of subjects known to suffer from bone marrow toxicity or a subject or group of subjects which has been brought into contact with at least one compound selected from the group consisting of Adriamycin hydrochloride, Carboplatin, Cisplatin, Cyclophosphamide monohydrate, Cytarabin, Ibuprofen, and Oxaliplatin, and said data processor executes instructions for comparing the amount of the at least one biomarker determined by the analyzing unit to the stored reference, wherein an essentially identical amount of the at least one biomarker in the test sample in comparison to the reference is indicative for the presence of bone marrow toxicity or wherein an amount of the at least one biomarker in the test sample which differs in comparison to the reference is indicative for the absence of bone marrow toxicity.

In another preferred embodiment of the device of the invention said stored reference is a reference derived from a subject or a group of subjects known to not suffer from bone marrow toxicity or a subject or group of subjects which has not been brought into contact with at least one compound selected from the group consisting of Adriamycin hydrochloride, Carboplatin, Cisplatin, Cyclophosphamide monohydrate, Cytarabin, Ibuprofen, and Oxaliplatin, and said data processor executes instructions for comparing the amount of the at least one biomarker determined by the analyzing unit to the stored reference, wherein an amount of the at least one biomarker in the test sample which differs in comparison to the reference is indicative for the presence of bone marrow toxicity or wherein an essential identical amount of the at least one biomarker in the test sample in comparison to the reference is indicative for the absence of bone marrow toxicity.

Further, the present invention relates to a kit for diagnosing bone marrow toxicity comprising a detection agent for the at least one biomarker selected from any one of Tables 1a, 1b, 1c, 1d, 1e, or 1f and standards for the at least one biomarker the concentration of which is derived from a subject or a group of subjects known to suffer from bone marrow toxicity or derived from a subject or a group of subjects known to not suffer from bone marrow toxicity.

In particular, the present invention relates to a method for diagnosing hematotoxicity comprising:
(a) determining the amount of at least one biomarker selected from any one of Tables 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9, 12 a or 12b in a test sample of a subject suspected to suffer from hematotoxicity, and
(b) comparing the amounts determined in step (a) to a reference, whereby hematotoxicity is to be diagnosed.

In a preferred embodiment of the aforementioned method said subject has been brought into contact with a compound suspected to be capable of inducing hematotoxicity.

The present invention also relates to a method of determining whether a compound is capable of inducing hematotoxicity in a subject comprising:
(a) determining in a sample of a subject which has been brought into contact with a compound suspected to be capable of inducing hematotoxicity the amount of at least one biomarker selected from any one of Tables 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9, 12a or 12b; and
(b) comparing the amounts determined in step (a) to a reference, whereby the capability of the compound to induce hematotoxicity is determined.

In a preferred embodiment of the aforementioned method said compound is at least one compound selected from the group consisting of: 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Saflufenacil, Cyclosporin A, Epoxiconazole, Flutamide, Lead acetate trihydrate, Linuron, Lithocholic acid, Methimazole, Methylprednisolone, Oxaliplatin, Probenecid, Tacrolimus and Triethanolamine.

In another preferred embodiment of the methods of the present invention said reference is derived from (i) a subject or group of subjects which suffers from hematotoxicity or (ii) a subject or group of subjects which has been brought into contact with at least one compound selected from the group consisting of: 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Saflufenacil, Cyclosporin A, Epoxiconazole, Flutamide, Lead acetate trihydrate, Linuron, Lithocholic acid, Methimazole, Methylprednisolone, Oxaliplatin, Probenecid, Tacrolimus and Triethanolamine. In a more preferred embodiment of said method essentially identical amounts for the biomarkers in the test sample and the reference are indicative for hematotoxicity.

In another preferred embodiment of the methods of the present invention said reference is derived from (i) a subject or group of subjects known to not suffer from hematotoxicity or (ii) a subject or group of subjects which has not been brought into contact with at least one compound selected from the group consisting of: 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Cyclosporin A, Epoxiconazole, Flutamide, Lead acetate trihydrate, Linuron, Lithocholic acid, Methimazole, Methylprednisolone, Oxaliplatin, Probenecid, Tacrolimus and Triethanolamine. In a more preferred embodiment of said methods amounts for the biomarkers which differ in the test sample in comparison to the reference are indicative for hematotoxicity.

In yet another embodiment of the methods of the present invention said reference is a calculated reference for the biomarkers for a population of subjects. In a more preferred embodiment of said methods amounts for the biomarkers which differ in the test sample in comparison to the reference are indicative for hematotoxicity.

The present invention also contemplates a method of identifying a substance for treating hematotoxicity comprising the steps of:
(a) determining in a sample of a subject suffering from hematotoxicity which has been brought into contact with a candidate substance suspected to be capable of treating hematotoxicity the amount of at least one biomarker selected from any one of Tables 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9, 12a or 12b; and
(b) comparing the amounts determined in step (a) to a reference, whereby a substance capable of treating hematotoxicity is to be identified.

In a preferred embodiment of the aforementioned method said reference is derived from (i) a subject or group of subjects which suffers from hematotoxicity or (ii) a subject or group of subjects which has been brought into contact with at least one compound selected from the group consisting of: 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Cyclosporin A, Epoxiconazole, Flutamide, Lead acetate trihydrate, Linuron, Lithocholic acid, Methimazole, Methylprednisolone, Oxaliplatin, Probenecid, Tacrolimus and Triethanolamine. In a more preferred embodiment of said method amounts for the biomarkers which differ in the test sample and the reference are indicative for a substance capable of treating hematotoxicity.

In another preferred embodiment of the aforementioned method said reference is derived from (i) a subject or group of subjects known to not suffer from hematotoxicity or (ii) a subject or group of subjects which has not been brought into contact with at least one compound selected from the group consisting of: 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Cyclosporin A, Epoxiconazole, Flutamide, Lead acetate trihydrate, Linuron, Lithocholic acid, Methimazole, Methylprednisolone, Oxaliplatin, Probenecid, Tacrolimus and Triethanolamine. In a more preferred embodiment of the said methods essentially identical amounts for the biomarkers in the test sample and the reference are indicative for a substance capable of treating hematotoxicity.

In yet another preferred embodiment of the aforementioned method said reference is a calculated reference for the biomarkers in a population of subjects. In a more preferred embodiment of the said methods essentially identical amounts for the biomarkers in the test sample and the reference are indicative for a substance capable of treating hematotoxicity.

The present invention also relates to the use of at least one biomarker selected from any one of Tables 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9, 12a or 12b or a detection agent for the said biomarker for diagnosing hematotoxicity in a sample of a subject.

Moreover, the present invention relates to a device for diagnosing hematotoxicity in a sample of a subject suspected to suffer therefrom comprising:
(a) an analyzing unit comprising a detection agent for at least one biomarker selected from any one of Tables 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9, 12a or 12b which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby hematotoxicity is diagnosed.

In a preferred embodiment of the device of the invention said stored reference is a reference derived from a subject or a group of subjects known to suffer from hematotoxicity or a subject or group of subjects which has been brought into contact with at least one compound selected from the group consisting of 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Cyclosporin A, Epoxiconazole, Flutamide, Lead acetate trihydrate, Linuron, Lithocholic acid, Methimazole, Methylprednisolone, Oxaliplatin, Probenecid, Tacrolimus and Triethanolamine, and said data processor executes instructions for comparing the amount of the at least one biomarker determined by the analyzing unit to the stored reference, wherein an essentially identical amount of the at least one biomarker in the test sample in comparison to the reference is indicative for the presence of hematotoxicity or wherein an amount of the at least one biomarker in the test sample which differs in comparison to the reference is indicative for the absence of hematotoxicity.

In another preferred embodiment of the device of the invention said stored reference is a reference derived from a subject or a group of subjects known to not suffer from hematotoxicity or a subject or group of subjects which has not been brought into contact with at least one compound selected from the group consisting of 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Cyclosporin A, Epoxiconazole, Flutamide, Lead acetate trihydrate, Linuron, Lithocholic acid, Methimazole, Methylprednisolone, Oxaliplatin, Probenecid, Tacrolimus and Triethanolamine, and said data processor executes instructions for comparing the amount of the at least one biomarker determined by the analyzing unit to the stored reference, wherein an amount of the at least one biomarker in the test sample which differs in comparison to the reference is indicative for the presence of hematotoxicity or wherein an essential identical amount of the at least one biomarker in the test sample in comparison to the reference is indicative for the absence of hematotoxicity.

Further, the present invention relates to a kit for diagnosing hematotoxicity comprising a detection agent for the at least one biomarker selected from any one of Tables 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9, 12a or 12b and standards for the at least one biomarker the concentration of which is derived from a subject or a group of subjects known to suffer from hematotoxicity or derived from a subject or a group of subjects known to not suffer from hematotoxicity.

In particular the present invention contemplates also the following specific methods, uses, devices and kits.

The following definitions and explanations apply mutatis mutandis to all the previous embodiments of the present invention as well as the embodiments described in the following.

The methods referred to in accordance with the present invention may essentially consist of the aforementioned steps or may include further steps. Further steps may relate to sample pre-treatment or evaluation of the diagnostic results obtained by the methods. Preferred further evaluation steps are described elsewhere herein. The methods may partially or entirely be assisted by automation. For example, steps pertaining to the determination of the amount of a biomarker can be automated by robotic and automated reader devices. Likewise, steps pertaining to a comparison of amounts can be automated by suitable data processing devices, such as a computer, comprising a program code which when being executed carries out the comparison automatically. A reference in such a case will be provided from a stored reference, e.g., from a database. It is to be understood that the method is, preferably, a method carried out ex vivo on a sample of a subject, i.e. not practised on the human or animal body.

The term “diagnosing”, as used herein refers to assessing the probability according to which a subject is suffering from a condition, such as intoxication, disease or disorder referred to herein, or has a predisposition for such a condition. Diagnosis of a predisposition may sometimes be referred to as prognosis or prediction of the likelihood that a subject will develop the condition within a predefined time window in the future. As will be understood by those skilled in the art, such an assessment, although preferred to be, may usually not be correct for 100% of the subjects to be diagnosed. The term, however, requires that a statistically significant portion of subjects can be identified as suffering from the condition or having a predisposition for the condition. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% or at least 95%. The p-values are, preferably, 0.2, 0.1, 0.05.

Diagnosing according to the present invention also includes monitoring, confirmation, and classification of a condition or its symptoms as well as a predisposition therefor. Monitoring refers to keeping track of an already diagnosed condition or predisposition. Monitoring encompasses, e.g., determining the progression of the condition or predisposition, determining the influence of a particular treatment on the progression of the condition or the influence of prophylactic measures such as a prophylactic treatment or diet on the development of the condition in a subject having a predisposition. Confirmation relates to the strengthening or substantiating a diagnosis of the condition or a predisposition for the condition already determined using other indicators or markers. Classification relates to (i) allocating the condition into different classes, e.g., corresponding to the strength of the symptoms accompanying the condition, or (ii) differentiating between different stages, disease or disorders accompanying the condition. A predisposition for the condition can be classified based on the degree of the risk, i.e. the probability according to which a subject will develop the condition later. Moreover, classification also, preferably, includes allocating a mode of action to a compound to be tested by the methods of the present invention. Specifically, the methods of the present invention allow for determination of a specific mode of action of a compound for which such mode of action is not yet known. This is, preferably, achieved by comparing the amount determined for the at least one biomarker or a biomarker profile representative for said compound to the amount of the biomarker or biomarker profile determined for a compound for which the mode of action is known as a reference. The classification of the mode of action allows an even more reliable assessment of toxicity of a compound because the molecular targets of the compound are identified.

The term “hematopoietic toxicity” as used herein relates to any damage or impairment of an organ or cells of the hematopoietic system which results in an impaired hematopoietic function, in particular, impaired hematopoiesis or impaired function of either erythrocytes or cells of the immune system such as the leucocytes. Preferably, affected by hematopoietic toxicity are the hematopoiesis in the bone marrow or the function of the immune system. Accordingly, the term hematopoietic toxicity as used herein encompasses bone marrow toxicity and hematotoxicity, in general. Preferably, hematopoietic toxicity as used herein is induced by or is the result of the administration of a chemical compound or drug, i.e. so-called toxin-induced hematopoietic toxicity.

The symptoms and clinical signs of the aforementioned manifestations of hematopoietic toxicity are well known to the person skilled in the art and are described in detail in standard books of toxicology, e.g., H. Marquardt, S. G. Schäfer, R. O. McClellan, F. Welsch (eds.), “Toxicology”, Chapter 13: The Liver, 1999, Academic Press, London.

Bone marrow toxicity as used herein refers, preferably, to an impairment of the function of the bone marrow. Preferably, bone marrow toxicity is accompanied by reduced proliferation or differentiation (lymphopoiesis) of pluripotent stem cells in the bone marrow. Bone marrow toxicity can be, preferably, accompanied by toxicity of rapidly proliferating marrow precursors or idiosyncratic marrow damage. Direct bone marrow damage may interfere with the marrow's ability to mount appropriate systemic responses. Alternatively, marrow damage may be reflected by maturation abnormalities in any or all of the proliferating marrow cell lines. This in turn can cause a variety of peripheral blood aberrations as well as morphologic abnormalities in the marrow. On, the other hand, when the marrow is the primary effector organ, proliferative responses in one or more cell lines may reflect an appropriate direct compound-related effect rather than a compensatory response to a systemic problem. In general, bone marrow toxicity and the accompanying marrow changes can be classified as either quantitative or qualitative. Quantitative abnormalities include the various hyperplasias and hypoplasias of the proliferating cell lines and require simultaneous evaluation of peripheral blood data for proper interpretation. Qualitative abnormalities refer to morphologic aberrations in marrow precursors (marrow dysplasias) as well as changes such as marrow necrosis, macrophage hyperplasia, and plasmacytosis. Bone marrow toxicity can be seen as a special type of maturation arrest in which both cytoplasm and nucleus may be affected. Systemic toxemia may affect the development of cells of all proliferating cell lines; however, toxicity is, preferably, most easily recognized in late-stage granulocyte precursors (metamyelocytes, band cells, and mature neutrophils). Bone marrow toxicity may be drug-induced, associated with circulating bacterial toxins in cases of severe infection, or caused by the circulating toxins released from sites of extensive tissue necrosis.

Preferably, the at least one biomarker to be determined by the methods of the present invention is selected from any one of Tables 1a, 1b, 1c, 1d, 1e, or 1f if the hematopoietic toxicity is bone marrow toxicity. More preferably, said bone marrow toxicity is bone marrow suppression, most preferably, bone marrow suppression inducible by platins, such as oxaliplatin.

Hematotoxicity as used herein, preferably, refers to an impairment of the function of the blood. Preferably, the function of the erythrocytes and/or the function of the leucocytes can be impaired. Preferably, hematotoxicity includes drug-induced aplastic anemia characterized by peripheral blood pancytopenia, reticulocytopenia, and bone marrow hypoplasia. Agents such as benzene and radiation have a predictable effect on hematopoietic progenitors, and the resulting aplastic anemia corresponds to the magnitude of the exposure to these agents. In contrast, idiosyncratic aplastic anemia does not appear to be related to the dose of the agent initiating the process. There are many agents which have been associated with the development of aplastic anemia, many of which have been reported in only a few patients. Aplastic, or non-regenerative, anemia is a syndrome associated with bone marrow failure, characterized by anemia, pancytopenia, and varying degrees of bone marrow hypocellularity. Aplastic anemia is classified as idiopathic or secondary, depending on whether its onset can be attributed to known causes, for example, ionizing radiation, drug, or chemical exposure. Aplastic anemia is a disorder of stem cell regulation, either through exhaustion of numbers, or a defect in differentiation, so that the stem cells are unable to recapitulate blood cells. Stromal cell defects may also play an important role in chronic bone marrow failure. In some of these cases there is evidence to support a clonal origin for aplastic anemia. Animal models of aplastic anemia are relatively few, and have been largely restricted to those induced by viruses, busulfan, irradiation, or benzene. The bone marrow has long been recognized as particularly susceptible to radiation-induced aplastic anemia in many species, including dogs, monkeys and mice. Aplastic anemia is also sometimes associated with exposure to drugs, including chloramphenicol, carbamazepine, felbamate, phenytoin, quinine, and phenylbutazone. Also, the term hematotoxicity includes lead toxicity. Lead has multiple hematologic effects among others, it decreases the ferrochelatase activity. This enzyme catalyzes the incorporation of the ferrous ion into the porphyrin ring structure. Failure to insert iron into protoporphyrin results in depressed heme formation. The excess protoporphyrin takes the place of heme in the hemoglobin molecule and, as the red blood cells containing protoporphyrin circulate, zinc is chelated at the center of the molecule at the site usually occupied by iron. Red blood cells containing zinc-protoporphyrin are intensely fluorescent and may be used to diagnose lead toxicity. Depressed heme synthesis is thought to be the stimulus for increasing the rate of activity of the first step in the heme synthetic pathway. Further, hematotoxicity may, preferably, affect platelets and/or platelet function. In particular, hematotoxicity may cause an impaired platelet response by causing thrombocytopenia or interfering with platelet function; some agents are capable of affecting both platelet number and function. Platelet function can be, preferably, determined by clotting assays for determining the coagulation function of the platelets. Thus, hematotoxicity as used herein, preferably, includes aplastic anemia, lead toxicity, inhibition of platelet aggregation and/or inhibition of porphyrin synthesis.

Preferably, the at least one biomarker to be determined by the methods of the present invention is selected from any one of Tables 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9, 12a or 12b if the hematopoietic toxicity is hematotoxicity.

More preferably, said hematotoxicity is characterized by blood anemia if the at least one biomarker is selected from the biomarkers shown in Table 2a, 2b, 12a or 12b. In particular, the biomarkers of table 12a and/or 12b were found to be early indicators of blood anemia. If rats were used as subjects in the methods of the invention, the said biomarkers were altered as early as 7 days after stimulation by any one of 2-Cloroaniline, aniline or 4-Chloro-3-nitroaniline.

More preferably, said hematotoxicity is characterized by an inhibition of the porphyrin synthesis if the at least one biomarker is selected from the biomarkers shown in Table 3a, 3b, 3c, 3d, 3e, 3f, or 3g.

Also more preferably, said hematotoxicity is characterized by an impaired methemoglobin level if the at least one biomarker is selected from the biomarkers shown in Table 4a, 4b, 4c, or 4d.

More preferably, said hematotoxicity is characterized by spleen heamosiderosis if the at least one biomarker is selected from the biomarkers shown in Table 5a, 5b, 5c, or 5d.

More preferably, said hematotoxicity is characterized by systemic impaired (anti-) proliferation of the cells of the hematopoietic system if the at least one biomarker is selected from the biomarkers shown in Table, 6a or 6b.

More preferably, said hematotoxicity is characterized by blood aplastic anemia if the at least one biomarker is selected from the biomarkers shown in Table 7a or 7b.

More preferably, said hematotoxicity is characterized by immunosuppression if the at least one biomarker is selected from the biomarkers shown in Table 8a or 8b.

More preferably, said hematotoxicity is characterized by spleen hematopoiesis if the at least one biomarker is selected from the biomarkers shown in Table 9.

It was found in accordance with the present invention that a combination of more than one of the biomarkers listed in the Tables further strengthen the diagnosis since each of the biomarkers is an apparently statistically independent predictor for the diagnosis. Moreover, the specificity for hematopoietic toxicity is also significantly increased since influences from other tissues on the marker abundance are counterbalanced. Thus, the term “at least one” as used herein, preferably; refers to a combination of at least 2, at least 3, at least 4, at least 5, at least 6, at least 7, at least 8, at least 9 or at least 10 of the biomarkers referred to in any one of the accompanying Tables. Preferably, all biomarkers recited in any one of the Tables are to be determined in combination in accordance with the methods of the present invention.

Preferred groups or combinations of biomarkers for hematopoietic toxicity from the individual tables and for the indications referred to in the tables are as follows:

Tables 1a, 1b: Progesterone, 4-Hydroxyphenylpyruvate, 21-Hydroxyprogesterone (11-Deoxycorticosterone), 18-Hydroxy-11-deoxycorticosterone or Citrate;

Tables 1c, 1d: Choline plasmalogen No 02, Valine, Leucine, Isoleucine or Ketoleucine

Tables 1e, 1f: Tryptophan, Ornithine, 14-Methylhexadecanoic acid, Glucose-6-phosphate or 18-Hydroxy-11-deoxycorticosterone

Tables 2a, 2b: Ribal, Cytosine, 18-Hydroxy-11-deoxycorticosterone, TAG (C16:0,C18:2) or TAG No 02

Tables 3a, 3b: Valine, Urea, Phenylalanine, Histidine or TAG (C16:0,C18:1,C18:3)

Tables 3c, 3d: Lysine, Sphingomyelin (d18:2,C18:0), Malate, DAG (C18:1,C18:2) or Isoleucine

Tables 3e, 3f: Isoleucine, Methionine, Leucine, Serine or Threonic acid

Table 3g: Isoleucine, Methionine, Phenylalanine, Leucine or Valine

Tables 4a, 4b: Serine, Ribal, Cytosine, Threonine or Docosahexaenoic acid (C22:cis[4,7,10,13,16,19]6)

Tables 4c, 4d: Threonine, Serine, Urea, Palmitoleic acid (C16:cis[9]1) or Glycine

Tables 5a, 5b: Linoleic acid (C18:cis[9,12]2), Docosahexaenoic acid (C22:cis[4,7,10,13,16,19]6), Heptadecanoic acid (C17:0), Phytosphingosine or Cytosine

Tables 5c, 5d: Ribal, Docosahexaenoic acid (C22:cis[4,7,10,13,16,19]6), Cytosine, Threonic acid or Palmitoleic acid (C16:cis[9]1)

Tables 6a, 6b: Coenzyme Q9, Coenzyme Q10, Cytosine, Mannose or Ribal

Tables 7a, 7b: gamma-Linolenic acid (C18:cis[6,9,12]3), Sphingomyelin (d18:1,C24:0), Histidine, Choline plasmalogen No 01 or Cytosine

Tables 8a, 8b: Cholesterolester No 01, Ketoleucine, Glutamate, Aspartate or 18-Hydroxy-11-deoxycorticosterone

Tables 9a, 9b: Uric acid, Cytosine, Uracil, Ascorbic acid or Ribal

Thus, preferably, the at least one biomarker is at least one biomarker selected from the aforementioned group or the at least one biomarker is a combination of biomarkers consisting or comprising the aforementioned group of biomarkers. The aforementioned biomarkers and combinations of biomarkers have been identified as key biomarkers having a particular high diagnostic value as described in more detail in the accompanying Examples.

Furthermore, other biomarkers or clinical parameters including known metabolites, genetic mutations, transcript and/or protein amounts or enzyme activities may still be determined in addition. Such, additional clinical or biochemical parameters which may be determined in accordance with the method of the present invention are well known in the art.

The term “biomarker” as used herein refers to a chemical compound whose presence or concentration in a sample is indicative for the presence or absence or strength of a condition, preferably, hematopoietic toxicity as referred to herein. The chemical compound is, preferably, a metabolite or an analyte derived therefrom. An analyte is a chemical compound which can be identical to the actual metabolite found in an organism. However, the term also includes derivatives of such metabolites which are either endogenously generated or which are generated during the isolation or sample pre-treatment or as a result of carrying out the methods of the invention, e.g., during the purification and/or determination steps. In specific cases the analyte is further characterized by chemical properties such as solubility. Due to the said properties, the analyte may occur in polar or lipid fractions obtained during the purification and/or determination process. Thus, chemical properties and, preferably, the solubility shall result in the occurrence of an analyte in either polar or lipid fractions obtained during the purification and/or determination process. Accordingly, the said chemical properties and, in particular the solubility taken into account as the occurrence of an analyte in either polar or lipid fractions obtained during the purification and/or determination process shall further characterize the analyte and assist in its identification. Details on how these chemical properties can be determined and taken into account are found in the accompanying Examples described below. Preferably, the analyte represents the metabolite in a qualitative and quantitative manner and, thus, allows inevitably concluding on the presence or absence or the amount of the metabolite in a subject or at least in the test sample of said subject. Biomarker, analyte and metabolite are referred to herein in the singular but also include the plurals of the terms, i.e. refer to a plurality of biomarker, analyte or metabolite molecules of the same molecular species. Moreover, a biomarker according to the present invention is not necessarily corresponding to one molecular species. Rather, the biomarker may comprise stereoisomers or enantiomers of a compound. Further, a biomarker can also represent the sum of isomers of a biological class of isomeric molecules. Said isomers shall exhibit identical analytical characteristics in some cases and are, therefore, not distinguishable by various analytical methods including those applied in the accompanying Examples described below. However, the isomers will share at least identical sum formula parameters and, thus, in the case of, e.g., lipids an identical chain length and identical numbers of double bonds in the fatty acid and/or sphingo base moieties

The term “test sample” as used herein refers to samples to be used for the diagnosis of hematopoietic toxicity by the methods of the present invention. Preferably, said test sample is a biological sample. Samples from biological sources (i.e. biological samples) usually comprise a plurality of metabolites. Preferred biological samples to be used in the method of the present invention are samples from body fluids, preferably, blood, plasma, serum, saliva, bile, urine or cerebrospinal fluid, or samples derived, e.g. by biopsy, from cells, tissues or organs, preferably from the liver. More preferably, the sample is a blood, plasma or serum sample, most preferably, a plasma sample. Biological samples are derived from a subject as specified elsewhere herein. Techniques for obtaining the aforementioned different types of biological samples are well known in the art. For example, blood samples may be obtained by blood taking while tissue or organ samples are to be obtained, e.g. by biopsy.

The aforementioned samples are, preferably, pre-treated before they are used for the methods of the present invention. As described in more detail below, said pre-treatment may include treatments required to release or separate the compounds or to remove excessive material or waste. Suitable techniques comprise centrifugation, extraction, fractioning, ultra-filtration, protein precipitation followed by filtration and purification and/or enrichment of compounds. Moreover, other pre-treatments are carried out in order to provide the compounds in a form or concentration suitable for compound analysis. For example, if gas-chromatography coupled mass spectrometry is used in the method of the present invention, it will be required to derivatize the compounds prior to the said gas chromatography. Suitable and necessary pre-treatments depend on the means used for carrying out the method of the invention and are well known to the person skilled in the art. Pre-treated samples as described before are also comprised by the term “sample” as used in accordance with the present invention.

The term “subject” as used herein relates to animals, preferably to mammals such as mice, rats, guinea pigs, rabbits, hamsters, pigs, sheep, dogs, cats, horses, monkeys, or cows and, also preferably, to humans. More preferably, the subject is a rodent and, most preferably, a rat. Other animals which may be diagnosed applying the methods of the present invention are fishes, birds or reptiles. Preferably, said subject was in or has been brought into contact with a compound suspected to be capable of inducing hematopoietic toxicity. A subject which has been brought into contact with a compound suspected to induce hematopoietic toxicity may, e.g., be a laboratory animal such as a rat which is used in a screening assay for, e.g., toxicity of compounds. A subject suspected to have been in contact with a compound capable of inducing hematopoietic toxicity may be also a subject to be diagnosed for selecting a suitable therapy. Preferably, a compound capable of inducing hematopoietic toxicity as used herein is 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Saflufenacil, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, or Ibuprofen.

Preferably, the at least one biomarker to be determined by the methods of the present invention is selected from any one of Tables 1a, 1b, 2a, 2b, 3a, 3b, 3c, 3d, 4a, 4b, 5a, 5b, 6a, or 6b if the subject is a female. A preferred group or combination of biomarkers for hematopoietic toxicity in a female subject is: 18-Hydroxy-11-deoxycorticosterone, 21-Hydroxyprogesterone (11-Deoxycorticosterone), 4-Hydroxyphenylpyruvate, Citrate, Coenzyme Q10, Coenzyme Q9, Cytosine, DAG (C18:1,C18:2), Docosahexaenoic acid (C22:cis[4,7,10,13,16,19]6), Heptadecanoic acid (C17:0), Histidine, Isoleucine, Linoleic acid (C18:cis[9,12]2), Lysine, Malate, Mannose, Phenylalanine, Phytosphingosine, Progesterone, Ribal, Serine, Sphingomyelin (d18:2,C18:0), TAG (C16:0,C18:1,C18:3), TAG (C16:0,C18:2), TAG No 02, Threonine, Urea, and Valine.

Preferably, the at least one biomarker to be determined by the methods of the present invention is selected from any one of Tables 1c, 1d, 1e, 1f, 3e, 3f, 3g, 4c, 4d, 5c, 5d, 7a, 7b, 8a, 8b, 9, 12a or 12b if the subject is a male. A preferred group or combination of biomarkers for hematopoietic toxicity in a male: 14-Methylhexadecanoic acid, 18-Hydroxy-11-deoxycorticosterone, Ascorbic acid, Aspartate, Cholesterolester No 01, Choline plasmalogen No 01, Choline plasmalogen No 02, Cytosine, Docosahexaenoic acid (C22:cis[4,7,10,13,16,19]6), gamma-Linolenic acid (C18:cis[6,9,12]3), Glucose-6-phosphate, Glutamate, Glycine, Histidine, Isoleucine, Ketoleucine, Leucine, Methionine, Omithine, Palmitoleic acid (C16:cis[9]1), Phenylalanine, Ribal, Serine, Sphingomyelin (d18:1,C24:0), Threonic acid, Threonine, Tryptophan, Uracil, Urea, Uric acid, and Valine.

The term “determining the amount” as used herein refers to determining at least one characteristic feature of the biomarker, i.e. the metabolite or analyte. Characteristic features in accordance with the present invention are features which characterize the physical and/or chemical properties including biochemical properties of a biomarker. Such properties include, e.g., molecular weight, viscosity, density, electrical charge, spin, optical activity, colour, fluorescence, chemoluminescence, elementary composition, chemical structure, capability to react with other compounds, capability to elicit a response in a biological read out system (e.g., induction of a reporter gene) and the like. Values for said properties may serve as characteristic features and can be determined by techniques well known in the art. Moreover, the characteristic feature may be any feature which is derived from the values of the physical and/or chemical properties of a biomarker by standard operations, e.g., mathematical calculations such as multiplication, division or logarithmic calculus. Most preferably, the at least one characteristic feature allows the determination and/or chemical identification of the biomarker and its amount. Accordingly, the characteristic value, preferably, also comprises information relating to the abundance of the biomarker from which the characteristic value is derived. For example, a characteristic value of a biomarker may be a peak in a mass spectrum. Such a peak contains characteristic information of the biomarker, i.e. the m/z (mass to charge ratio) information, as well as an intensity value being related to the abundance of the said biomarker (i.e. its amount) in the sample.

As discussed before, the at least one biomarker to be determined in accordance with the methods of the present invention may be, preferably, determined quantitatively or semi-quantitatively. For quantitative determination, either the absolute or precise amount of the biomarker will be determined or the relative amount of the biomarker will be determined based on the value determined for the characteristic feature(s) referred to herein above. The relative amount may be determined in a case were the precise amount of a biomarker can or shall not be determined. In said case, it can be determined whether the amount in which the biomarker is present is enlarged or diminished with respect to a second sample comprising said biomarker in a second amount. Quantitatively analysing a biomarker, thus, also includes what is sometimes referred to as semi-quantitative analysis of a biomarker.

Moreover, determining as used in the methods of the present invention, preferably, includes using a compound separation step prior to the analysis step referred to before. Preferably, said compound separation step yields a time resolved separation of the at least one biomarker comprised by the sample. Suitable techniques for separation to be used preferably in accordance with the present invention, therefore, include all chromatographic separation techniques such as liquid chromatography (LC), high performance liquid chromatography (HPLC), gas chromatography (GC), thin layer chromatography, size exclusion or affinity chromatography. These techniques are well known in the art and can be applied by the person skilled in the art without further ado. Most preferably, LC and/or GC are chromatographic techniques to be envisaged by the methods of the present invention. Suitable devices for such determination of biomarkers are well known in the art. Preferably, mass spectrometry is used in particular gas chromatography mass spectrometry (GC-MS), liquid chromatography mass spectrometry (LC-MS), direct infusion mass spectrometry or Fourier transform ion-cyclotrone-resonance mass spectrometry (FT-ICR-MS), capillary electrophoresis mass spectrometry (CE-MS), high-performance liquid chromatography coupled mass spectrometry (HPLC-MS), quadrupole mass spectrometry, any sequentially coupled mass spectrometry, such as MS-MS or MS-MS-MS, inductively coupled plasma mass spectrometry (ICP-MS), pyrolysis mass spectrometry (Py-MS), ion mobility mass spectrometry or time of flight mass spectrometry (TOF). Most preferably, LC-MS and/or GC-MS are used as described in detail below. Said techniques are disclosed in, e.g., Nissen 1995, Journal of Chromatography A, 703: 37-57, U.S. Pat. No. 4,540,884 or U.S. Pat. No. 5,397,894, the disclosure content of which is hereby incorporated by reference. As an alternative or in addition to mass spectrometry techniques, the following techniques may be used for compound determination: nuclear magnetic resonance (NMR), magnetic resonance imaging (MRI), Fourier transform infrared analysis (FT-IR), ultraviolet (UV) spectroscopy, refraction index (RI), fluorescent detection, radiochemical detection, electrochemical detection, light scattering (LS), dispersive Raman spectroscopy or flame ionisation detection (FID). These techniques are well known to the person skilled in the art and can be applied without further ado. The method of the present invention shall be, preferably, assisted by automation. For example, sample processing or pre-treatment can be automated by robotics. Data processing and comparison is, preferably, assisted by suitable computer programs and databases. Automation as described herein before allows using the method of the present invention in high-throughput approaches.

Moreover, the biomarker can also be determined by a specific chemical or biological assay. Said assay shall comprise means which allow for specifically detecting the biomarker in the sample. Preferably, said means are capable of specifically recognizing the chemical structure of the biomarker or are capable of specifically identifying the biomarker based on its capability to react with other compounds or its capability to elicit a response in a biological read out system (e.g., induction of a reporter gene). Means which are capable of specifically recognizing the chemical structure of a biomarker are, preferably, detection agents which specifically bind to the biomarker, more preferably, antibodies or other proteins which specifically interact with chemical structures, such as receptors or enzymes, or aptameres. Specific antibodies, for instance, may be obtained using the biomarker as antigen by methods well known in the art. Antibodies as referred to herein include both polyclonal and monoclonal antibodies, as well as fragments thereof, such as Fv, Fab and F(ab)₂ fragments that are capable of binding the antigen or hapten. The present invention also includes humanized hybrid antibodies wherein amino acid sequences of a non-human donor antibody exhibiting a desired antigen-specificity are combined with sequences of a human acceptor antibody. Moreover, encompassed are single chain antibodies. The donor sequences will usually include at least the antigen-binding amino acid residues of the donor but may comprise other structurally and/or functionally relevant amino acid residues of the donor antibody as well. Such hybrids can be prepared by several methods well known in the art. Suitable proteins which are capable of specifically recognizing the metabolite are, preferably, enzymes which are involved in the metabolic conversion of the said biomarker. Said enzymes may either use the biomarker, e.g., a metabolite, as a substrate or may convert a substrate into the biomarker, e.g., metabolite. Moreover, said antibodies may be used as a basis to generate oligopeptides which specifically recognize the biomarker. These oligopeptides shall, for example, comprise the enzyme's binding domains or pockets for the said biomarker. Suitable antibody and/or enzyme based assays may be RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA) or solid phase immune tests. Aptameres which specifically bind to the biomarker can be generated by methods well known in the art (Ellington 1990, Nature 346:818-822; Vater 2003, Curr Opin Drug Discov Devel 6(2): 253-261). Moreover, the biomarker may also be identified based on its capability to react with other compounds, i.e. by a specific chemical reaction. Further, the biomarker may be determined in a sample due to its capability to elicit a response in a biological read out system. The biological response shall be detected as read out indicating the presence and/or the amount of the metabolite comprised by the sample. The biological response may be, e.g., the induction of gene expression or a phenotypic response of a cell or an organism.

The term “reference” refers to values of characteristic features of the at least one biomarker and, preferably, values indicative for an amount of the said biomarker which can be correlated to hematopoietic toxicity.

Such references are, preferably, obtained from a sample derived from a subject or group of subjects which suffer from hematopoietic toxicity or from a sample derived from a subject or group of subjects which have/has been brought into contact with 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Saflufenacil, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, or Ibuprofen. A subject or group of subjects may be brought into contact with the said compounds by each topic or systemic administration mode as long as the compounds become bioavailable.

Preferably, the aforementioned compounds can be administered to the subject or the individuals of the group of subjects from which the reference is derived as described in the accompanying Examples and Tables below.

In particular, Adriamycin hydrochloride, Carboplatin, Cisplatin, Cyclophosphamide monohydrate, Cytarabin, Ibuprofen, and Oxaliplatin as referred to herein are compounds capable of inducing bone marrow toxicity while 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Saflufenacil, Cyclosporin A, Epoxiconazole, Flutamide, Lead acetate trihydrate, Linuron, Lithocholic acid, Methimazole, Methylprednisolone, Oxaliplatin, Probenecid, Tacrolimus or Triethanolamine shall be capable of inducing hematotoxicity.

Alternatively, but nevertheless also preferred, the reference may be obtained from sample derived from a subject or group of subjects which has not been brought into contact with 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Saflufenacil, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, or Ibuprofen or a healthy subject or group of such subjects with respect to hematopoietic toxicity and, more preferably, other diseases as well.

The reference may be determined as described hereinabove for the amounts of the biomarkers. In particular, a reference is, preferably, obtained from a sample of a group of subjects as referred to herein by determining the relative or absolute amounts of each of the at least one biomarker(s) in samples from each of the individuals of the group separately and subsequently determining a median or average value for said relative or absolute amounts or any parameter derived therefrom by using statistical techniques referred to elsewhere herein. Alternatively, the reference may be, preferably, obtained by determining the relative or absolute amount for each of the at least one biomarker in a sample from a mixture of samples of the group of subjects as referred to herein. Such a mixture, preferably, consists of portions of equal volume from samples obtained from each of the individuals of the said group.

Moreover, the reference, also preferably, could be a calculated reference, most preferably the average or median value, for the relative or absolute amount for each of the at least one biomarker derived from a population of individuals. Said population of individuals is the population from which the subject to be investigated by the method of the present invention originates. However, it is to be understood that the population of subjects to be investigated for determining a calculated reference, preferably, either consist of apparently healthy subjects (e.g. untreated) or comprise a number of apparently healthy subjects which is large enough to be statistically resistant against significant average or median changes due to the presence of the test subject(s) in the said population. The absolute or relative amounts of the at least one biomarker of said individuals of the population can be determined as specified elsewhere herein. How to calculate a suitable reference value, preferably, the average or median, is well known in the art. Other techniques for calculating a suitable reference include optimization using receiver operating characteristics (ROC) curve calculations which are also well known in the art and which can be performed for an assay system having a given specificity and sensitivity based on a given cohort of subjects without further ado. The population or group of subjects referred to before shall comprise a plurality of subjects, preferably, at least 5, 10, 50, 100, 1,000 or 10,000 subjects up to the entire population. More preferably, the group of subjects referred to in this context is a group of subjects having a size, being statistically representative for a given population, i.e. a statistically representative sample. It is to be understood that the subject to be diagnosed by the methods of the present invention and the subjects of the said plurality of subjects are of the same species and, preferably, of the same gender.

More preferably, the reference will be stored in a suitable data storage medium such as a database and are, thus, also available for future diagnoses. This also allows efficiently diagnosing predisposition for hematopoietic toxicity because suitable reference results can be identified in the database once it has been confirmed (in the future) that the subject from which the corresponding reference sample was obtained (indeed) developed hematopoietic toxicity.

The term “comparing” refers to assessing whether the amount of the qualitative or quantitative determination of the at least one biomarker is identical to a reference or differs therefrom.

In case the reference results are obtained from a sample derived from a subject or group of subjects suffering from hematopoietic toxicity or a subject or group of subjects which has been brought into contact with 1,3-Dinitrobenzene, 1,4-Dinitrobenzene, 2-Butoxyethanol, 2-Chloroaniline, Cyclohexanone oxime (CHO), 4-Chloro-3-nitroaniline, Aniline, Saflufenacil, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Adriamycin hydrochloride, Carboplatin, Cisplatin, Cyclophosphamide monohydrate, Cytarabin, or Ibuprofen, hematopoietic toxicity can be diagnosed based on the degree of identity or similarity between the amounts obtained from the test sample and the aforementioned reference, i.e. based on an identical qualitative or quantitative composition with respect to the at least one biomarker. Identical amounts include those amounts which do not differ in a statistically significant manner and are, preferably, within at least the interval between 1st and 99th percentile, 5th and 95th percentile, 10th and 90th percentile, 20th and 80th percentile, 30th and 70th percentile, 40th and 60th percentile of the reference, more preferably, the 50th, 60th, 70th, 80th, 90th or 95th percentile of the reference. A reference obtained from a sample derived from a subject or group of subjects suffering from hematopoietic toxicity or a subject or group of subjects which has been brought into contact with 1,3-Dinitrobenzene, 1,4-Dinitrobenzene, 2-Butoxyethanol, 2-Chloroaniline, Cyclohexanone oxime (CHO), 4-Chloro-3-nitroaniline, Aniline, Saflufenacil, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Adriamycin hydrochloride, Carboplatin, Cisplatin, Cyclophosphamide monohydrate, Cytarabin, or Ibuprofen, can be applied in the methods of the present invention in order to diagnose hematopoietic toxicity or for determining whether a compound is capable of inducing hematopoietic toxicity in a subject. In such a case, preferably, an amount of the at least one biomarker which is essentially identical to the reference will be indicative for the presence of hematopoietic toxicity or a compound which is capable of inducing hematopoietic toxicity, while an amount of the at least one biomarker which differs from the reference will be indicative for the absence of hematopoietic toxicity or a compound which is not capable of inducing hematopoietic toxicity.

Moreover, a reference obtained from a sample derived from a subject or group of subjects suffering from hematopoietic toxicity or a subject or group of subjects which has been brought into contact with 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Saflufenacil, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, or Ibuprofen, can be applied for identifying a substance for treating hematopoietic toxicity. In such a case, preferably, an amount of the at least one biomarker which differs from the reference will be indicative for a substance suitable for treating hematopoietic toxicity, while an amount of the at least one biomarker which is essentially identical to the reference will be indicative for a substance which is not capable of treating hematopoietic toxicity.

In case the reference results are obtained from a sample of a subject or group of subjects which has not been brought into contact with 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Saflufenacil, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, or Ibuprofen or which does not suffer from hematopoietic toxicity, said hematopoietic toxicity can be diagnosed based on the differences between the test amounts obtained from the test sample and the aforementioned reference, i.e. differences in the qualitative or quantitative composition with respect to the at least one biomarker.

The same applies if a calculated reference as specified above is used.

The difference may be an increase in the absolute or relative amount of the at least one biomarker (sometimes referred to as up-regulation of the biomarker; see also Examples) or a decrease in either of said amounts or the absence of a detectable amount of the biomarker (sometimes referred to as down-regulation of the biomarker; see also Examples). Preferably, the difference in the relative or absolute amount is significant, i.e. outside of the interval between 45^{th} and 55^{th} percentile, 40^{th} and 60^{th} percentile, 30^{th} and 70^{th} percentile, 20^{th} and 80^{th} percentile, 10^{th} and 90^{th} percentile, 5th and 95^{th} percentile, 1st and 99^{th} percentile of the reference.

A reference obtained from a sample derived from a subject or group of subjects which has not been brought into contact with 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Saflufenacil, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, or Ibuprofen or which does not suffer from hematopoietic toxicity can be applied in the methods of the present invention in order to diagnose the hematopoietic toxicity or for determining whether a compound is capable of inducing hematopoietic toxicity in a subject. In such a case, preferably, an amount of the at least one biomarker which differs from the reference will be indicative for the presence of hematopoietic toxicity or a compound which is capable of inducing hematopoietic toxicity, while an amount of the at least one biomarker which is essentially identical to the reference will be indicative for the absence of hematopoietic toxicity or a compound which is not capable of inducing hematopoietic toxicity. Moreover, a reference obtained from a sample derived from a subject or group of subjects which has not been brought into contact with 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Saflufenacil, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanoamine, Carboplatin, Cisplatin, Cyclophosphamide monohydrate, or Cytarabin, or which does not suffer from hematopoietic toxicity can be applied for identifying a substance for treating hematopoietic toxicity. In such a case, preferably, an amount of the at least one biomarker which is essentially identical to the reference will be indicative for a substance suitable for treating hematopoietic toxicity, while an amount of the at least one biomarker which differs from the reference will be indicative for a substance which is not suitable for treating hematopoietic toxicity.

Preferred references are those referred to in the accompanying Tables or those which can be generated following the accompanying Examples. Moreover, relative differences, i.e. increases or decreases in the amounts for individual biomarkers, are preferably, those recited in the Tables below. Moreover, preferably, the extent of an observed difference, i.e. an increase or decrease, is preferably, an increase or decrease according to the factor indicated in the Tables, below.

Preferably, the at least one biomarker when selected from Tables 1a, 1c, 1e, 2a, 3a, 3c, 3e, 4a, 4c, 5a, 5c, 6a, 7a, 8a or 12b is increased with respect to a reference obtained from a sample derived from a subject or group of subjects which has not been brought into contact with 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, or Ibuprofen or a sample obtained from a healthy subject or group of subjects as indicated in the said Tables.

Preferably, the at least one biomarker when selected from Tables 1b, 1d, 1f, 2b, 3b, 3d, 3f, 3g, 4b, 4d, 5b, 5d, 6b, 7b, 8b, 9 or 12a is decreased with respect to a reference obtained from a sample derived from a subject or group of subjects which has not been brought into contact with 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, or Ibuprofen or a sample obtained from a healthy subject or group of subjects as indicated in the said Tables.

The comparison is, preferably, assisted by automation. For example, a suitable computer program comprising algorithm for the comparison of two different data sets (e.g., data sets comprising the values of the characteristic feature(s)) may be used. Such computer programs and algorithm are well known in the art. Notwithstanding the above, a comparison can also be carried out manually.

The term “substance for treating hematopoietic toxicity” refers to compounds which may directly interfere with the biological mechanisms inducing hematopoietic toxicity referred to elsewhere in this specification Alternatively, but also preferred the compounds may interfere with the development or progression of symptoms associated with the hematopoietic toxicity. Substances to be identified by the method of the present invention may be organic and inorganic chemicals, such as small molecules, polynucleotides, oligonucleotides including siRNA, ribozymes or micro RNA molecules, peptides, polypeptides including antibodies or other artificial or biological polymers, such as aptameres. Preferably, the substances are suitable as drugs, pro-drugs or lead substances for the development of drugs or pro-drugs.

It is to be understood that if the methods of the present invention are to be used for identifying drugs for the therapy of hematopoietic toxicity or for toxicological assessments of compounds (i.e. determining whether a compound is capable of inducing hematopoietic toxicity), test samples of a plurality of subjects may be investigated for statistical reasons. Preferably, the metabolome within such a cohort of test subjects shall be as similar as possible in order to avoid differences which are caused, e.g., by factors other than the compound to be investigated. Subjects to be used for the said methods are, preferably, laboratory animals such as rodents and more preferably rats. It is to be understood further that the said laboratory animals shall be, preferably, sacrificed after completion of the methods of the present invention. All subjects of a cohort test and reference animals shall be kept under identical conditions to avoid any differential environmental influences. Suitable conditions and methods of providing such animals are described in detail in WO2007/014825. Said conditions are hereby incorporated by reference.

The methods of the present invention can be, preferably, implemented by the device of the present invention. A device as used herein shall comprise at least the aforementioned units. The units of the device are operatively linked to each other. How to link the units in an operating manner will depend on the type of units included into the device. For example, where means for automatically qualitatively or quantitatively determining the at least one biomarker are applied in an analyzing unit, the data obtained by said automatically operating unit can be processed by the evaluation unit, e.g., by a computer program which runs on a computer being the data processor in order to facilitate the diagnosis. Preferably, the units are comprised by a single device in such a case. However, the analyzing unit and the evaluation unit may also be physically separate. In such a case operative linkage can be achieved via wire and wireless connections between the units which allow for data transfer. A wireless connection may use Wireless LAN (WLAN) or the Internet. Wire connections may be achieved by optical and non-optical cable connections between the units. The cables used for wire connections are, preferably, suitable for high throughput data transport

A preferred analyzing unit for determining at least one biomarker comprises a detection agent, such as an antibody, protein or aptamere which specifically recognizes the at least one biomarker as specified elsewhere herein, and a zone for contacting said detection agent with the sample to be tested. The detection agent may be immobilized on the zone for contacting or may be applied to the said zone after the sample has been loaded. The analyzing unit shall be, preferably, adapted for qualitatively and/or quantitatively determine the amount of complexes of the detection agent and the at least one biomarker. It will be understood that upon binding of the detection agent to the at least one biomarker, at least one measurable physical or chemical property of either the at least one biomarker, the detection agent or both will be altered such that the said alteration can be measured by a detector, preferably, comprised in the analyzing unit. However, where analyzing units such as test stripes are used, the detector and the analyzing units may be separate components which are brought together only for the measurement. Based on the detected alteration in the at least one measurable physical or chemical property, the analyzing unit may calculate an intensity value for the at least one biomarker as specified elsewhere herein. Said intensity value can then be transferred for further processing and evaluation to the evaluation unit. Most preferably, the amount of the at least one biomarker can be determined by ELISA, EIA, or RIA based techniques using a detection agent as specified elsewhere herein. Alternatively, an analyzing unit as referred to herein, preferably, comprises means for separating biomarkers, such as chromatographic devices, and means for biomarker determination, such as spectrometry devices. Suitable devices have been described in detail above. Preferred means for compound separation to be used in the system of the present invention include chromatographic devices, more preferably devices for liquid chromatography, HPLC, and/or gas chromatography. Preferred devices for compound determination comprise mass spectrometry devices, more preferably, GC-MS, LC-MS, direct infusion mass spectrometry, FT-ICR-MS, CE-MS, HPLC-MS, quadrupole mass spectrometry, sequentially coupled mass spectrometry (including MS-MS or MS-MS-MS), ICP-MS, Py-MS or TOF. The separation and determination means are, preferably, coupled to each other. Most preferably, LC-MS and/or GC-MS is used in the analyzing unit referred to in accordance with the present invention.

The evaluation unit of the device of the present invention, preferably, comprises a data processing device or computer which is adapted to execute rules for carrying out the comparison as specified elsewhere herein. Moreover, the evaluation unit, preferably, comprises a database with stored references. A database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative for hematopoietic toxicity (e.g. a query search). Thus, if an identical or similar data set can be identified in the data collection, the test data set will be associated with hematopoietic toxicity. The evaluation unit may also preferably comprise or be operatively linked to a further database with recommendations for therapeutic or preventive interventions or life style adaptations based on the established diagnosis of hematopoietic toxicity. Said further database can be, preferably, automatically searched with the diagnostic result obtained by the evaluation unit in order to identify suitable recommendations for the subject from which the test sample has been obtained in order to treat or prevent hematopoietic toxicity.

In a preferred embodiment of the device of the present invention, said stored reference is a reference derived from a subject or a group of subjects known to suffer from hematopoietic toxicity or a subject or group of subjects which has been brought into contact with at least one compound selected from the group consisting of 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Saflufenacil, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen, and said data processor executes instructions for comparing the amount of the at least one biomarker determined by the analyzing unit to the stored reference, wherein an essentially identical amount of the at least one biomarker in the test sample in comparison to the reference is indicative for the presence of hematopoietic toxicity or wherein an amount of the at least one biomarker in the test sample which differs in comparison to the reference is indicative for the absence of hematopoietic toxicity.

In another preferred embodiment of the device of the present invention, said stored reference is a reference derived from a subject or a group of subjects known not to suffer from hematopoietic toxicity or a subject or group of subjects which has not been brought into contact with at least one compound selected from the group consisting of 1,3-Dinitrobenzene, 1,4-Dinitrobenzerie, 2-Butoxyethanol, 2-Chloroaniline, Cyclohexanone oxime (CHO), 4-Chloro-3-nitroaniline, Adriamycin hydrochloride, Aniline, Saflufenacil, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen, and said data processor executes instructions for comparing the amount of the at least one biomarker determined by the analyzing unit to the stored reference, wherein an amount of the at least one biomarker in the test sample which differs in comparison to the reference is indicative for the presence of hematopoietic toxicity or wherein an essentially identical amount of the at least one biomarker in the test sample in comparison to the reference is indicative for the absence of hematopoietic toxicity.

The device, thus, can also be used without special medical knowledge by medicinal or laboratory staff or patients, in particular when an expert system making recommendations is included. The device is also suitable for near-patient applications since the device can be adapted to a portable format.

The term “kit” refers to a collection of the aforementioned components, preferably, provided separately or within a single container. The container also comprises instructions for carrying out the method of the present invention. These instructions may be in the form of a manual or may be provided by a computer program code which is capable of carrying out the comparisons referred to in the methods of the present invention and to establish a diagnosis accordingly when implemented on a computer or a data processing device. The computer program code may be provided on a data storage medium or device such as an optical or magnetic storage medium (e.g., a Compact Disc (CD), CD-ROM, a hard disk, optical storage media, or a diskette) or directly on a computer or data processing device. A “standard” as referred to in connection with the kit of the invention is an amount of the at least one biomarker when present in solution or dissolved in a predefined volume of a solution resembles the amount of the at least one biomarker which is present (i) in a subject or a group of subjects known to suffer from hematopoietic toxicity or a subject or group of subjects which has been brought into contact with at least one compound selected from the group consisting of 1,3-Dinitrobenzene, 1,4-Dinitrobenzene; 2-Butoxyethanol, 2-Chloroaniline, Cyclohexanone oxime (CHO), 4-Chloro-3-nitroaniline, Adriamycin hydrochloride, Aniline, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen or (ii) derived from a subject or a group of subjects known to not suffer from therefrom or a subject or group of subjects which has not been brought into contact with at least one compound selected from the group consisting of 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen.

Advantageously, it has been found in the study underlying the present invention that the amount of at least one biomarker as specified herein allows for diagnosing hematopoietic toxicity, specifically hematopoietic toxicity induced by 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen. The specificity and accuracy of the method will be even more improved by determining an increasing number or even all of the aforementioned biomarkers. A change in the quantitative and/or qualitative composition of the metabolome with respect to these specific biomarkers is indicative for hematopoietic toxicity even before other signs of the said toxicity are clinically apparent. The morphological, physiological as well as biochemical parameters which are currently used for diagnosing hematopoietic toxicity are less specific and less sensitive in comparison to the biomarker determination provided by the present invention. Thanks to the present invention, hematopoietic toxicity of a compound can be more efficiently and reliably assessed. Moreover, based on the aforementioned findings, screening assays for drugs which are useful for the therapy of hematopoietic toxicity are feasible. In general, the present invention contemplates the use of at least one biomarker in a sample of a subject selected from any one of the Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9, 12a or 12b or a detection agent for said biomarker for diagnosing hematopoietic toxicity, for determining whether a compound is capable of inducing hematopoietic toxicity or for identifying a substance capable of treating hematopoietic toxicity. Further, the present invention, in general, contemplates the use of the at least one biomarker in a sample of a subject or a detection agent therefor for identifying a subject being susceptible for a treatment of hematopoietic toxicity. Preferred detection agents to be used in this context of the invention are those referred to elsewhere herein. Moreover, the methods of the present invention can be, advantageously, implemented into a device. Furthermore, a kit can be provided which allows for carrying out the methods.

The present invention also relates to a data collection comprising characteristic values for the biomarkers recited in any one of Tables 1a, 1b, 1c, 1d; 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9, 12a or 12b. The term “data collection” refers to a collection of data which may be physically and/or logically grouped together. Accordingly, the data collection may be implemented in a single data storage medium or in physically separated data storage media being operatively linked to each other. Preferably, the data collection is implemented by means of a database. Thus, a database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative for hematopoietic toxicity (e.g. a query search). Thus, if an identical or similar data set can be identified in the data collection, the test data set will be associated with hematopoietic toxicity. Consequently, the information obtained from the data collection can be used to diagnose hematopoietic toxicity based on a test data set obtained from a subject.

Moreover, the present invention pertains to a data storage medium comprising the said data collection. The term “data storage medium” as used herein encompasses data storage media which are based on single physical entities such as a CD, a CD-ROM, a hard disk, optical storage media, or a diskette. Moreover, the term further includes data storage media consisting of physically separated entities which are operatively linked to each other in a manner as to provide the aforementioned data collection, preferably, in a suitable way for a query search.

The present invention also relates to a system comprising
(a) means for comparing characteristic values of at least one biomarker of a sample operatively linked to
(b) the data storage medium of the present invention.

The term “system” as used herein relates to different means which are operatively linked to each other. Said means may be implemented in a single device or may be implemented in physically separated devices which are operatively linked to each other. The means for comparing characteristic values of the biomarker operate, preferably, based on an algorithm for comparison as mentioned before. The data storage medium, preferably, comprises the aforementioned data collection or database, wherein each of the stored data sets being indicative for hematopoietic toxicity. Thus, the system of the present invention allows identifying whether a test data set is comprised by the data collection stored in the data storage medium. Consequently, the system of the present invention may be applied as a diagnostic means in diagnosing hematopoietic toxicity. In a preferred embodiment of the system, means for determining characteristic values of biomakers of a sample are comprised. The term “means for determining characteristic values of biomarkers” preferably relates to the aforementioned devices for the determination of biomarkers such as mass spectrometry devices, ELISA devices, NMR devices or devices for carrying out chemical or biological assays for the analytes.

All references referred to above are herewith incorporated by reference with respect to their entire disclosure content as well as their specific disclosure content explicitly referred to in the above description.

### FIGURES

The following Examples are merely for the purposes of illustrating the present invention. They shall not be construed, whatsoever, to limit the scope of the invention in any respect.

### EXAMPLES

### Example

### Biomarkers Associated with Hematopoietic Toxicity

A group of each 5 male and female rats was dosed once daily with the indicated compounds (see Table 10, below for compounds, applied doses and administration details) over 28 days.

Each dose group in the studies consisted of five rats per sex. Additional groups of each 5 male and female animals served as controls. Before starting the treatment period, animals, which were 62-64 days old when supplied, were acclimatized to the housing and environmental conditions for 7 days. All animals of the animal population were kept under the same constant temperature (20-24±3° C.) and the same constant humidity (30-70%). The animals of the animal population were fed ad libitum. The food to be used was essentially free of chemical or microbial contaminants. Drinking water was also offered ad libitum. Accordingly, the water was free of chemical and microbial contaminants as laid down in the European Drinking Water Directive 98/83/EG. The illumination period was 12 hours light followed by 12 hours darkness (12 hours light, from 6:00 to 18:00, and 12 hours darkness, from 18:00 to 6:00). The studies were performed in an AAALAC-approved laboratory in accordance with the German Animal Welfare Act and the European Council Directive 86/609/EE. The test system was arranged according to the OECD 407 guideline for the testing of chemicals for repeated dose 28-day oral toxicity study, in rodents. The test substances (compounds) in the Tables 1 to 9 below were dosed and administered as described in the Table 10 above.

In the morning of day 7, 14, and 28, blood was taken from the retroorbital venous plexus from fasted anaesthetized animals. From each animal, 1 ml of blood was collected with EDTA as anticoagulant. The samples were centrifuged for generation of plasma. All plasma samples were covered with a N2 atmosphere and then stored at −80° C. until analysis.

For mass spectrometry-based metabolite profiling analyses plasma samples were extracted and a polar and a non-polar (lipid) fraction was obtained. For GC-MS analysis, the non-polar fraction was treated with methanol under acidic conditions to yield the fatty acid methyl esters. Both fractions were further derivatised with O-methyl-hydroxyamine hydrochloride and pyridine to convert Oxo-groups to O-methyloximes and subsequently with a silylating agent before analysis. In LC-MS analysis, both fractions were reconstituted in appropriate solvent mixtures. HPLC was performed by gradient elution on reversed phase separation columns. Mass spectrometric detection which allows target and high sensitivity MRM (Multiple Reaction Monitoring) profiling in parallel to a full screen analysis was applied as described in WO2003073464.

Steroids and their metabolites were measured by online SPE-LC-MS (Solid phase extraction-LC-MS). Catecholamines and their metabolites were measured by online SPE-LC-MS as described by Yamada et al. (Yamada 2002, Journal of Analytical Toxicology, 26(1): 17-22))

Following comprehensive analytical validation steps, the data for each analyte were normalized against data from pool samples. These samples were run in parallel through the whole process to account for process variability. The significance of treatment group values specific for sex, treatment-duration and metabolite was determined by comparing means of the treated groups to the means of the respective untreated control groups using WELCH-test and quantified with treatment ratios versus control and p-values.

The identification of the most important biomarkers per toxicity pattern was done by a ranking of the analytes in the tables below. Therefore the metabolic changes in reference treatments of a given pattern (shown in the table) were compared with changes of the same metabolite in other unrelated treatments. For each metabolite T-values were obtained for the reference and control treatment and compared by the Welch test to asses whether these two groups are significantly different. The maximum absolute value of the respective TVALUE was taken to indicate the most important metabolite for the pattern.

The changes of the group of plasma metabolites being indicative for hematopoietic toxicity after treatment of the rats are shown in the following tables:

TABLE 1a
Markers for hematotoxicity in female rats; Significant
up-regulation changes (p-Value ≦ 0.2) are marked
(*). For some metabolites (marked with #), additional
information are provided in table 11. Bone marrow suppression
inducing Oxaliplatin was administered at low dose.

| | Carbo- | Cis- | Ibu- | |
|---|---|---|---|---|
| | platin | platin | profen | Oxaliplatin |
| Metabolite | f28 | f28 | f28 | f28 |
| Leucine | 1.27 * | 1.3 * | 1.25 * | 1.35 * |
| Isoleucine | 1.18 * | 1.28 * | 1.22 * | 1.39 * |
| Valine | 1.13 * | 1.23 | 1.23 * | 1.22 * |
| Aspartate | 1.58 * | 1.14 | 1.08 * | 2.09 * |
| Phosphate | 1.57 * | 1.25 * | 1.58 * | 1.53 * |
| (inorganic and or- | | | | |
| ganic phosphates) | | | | |
| Glycerol-3-phosphate, | 1.94 * | 1.2 * | 1.27 * | 1.28 * |
| polar fraction | | | | |
| Phosphatidylcholine | 1.22 * | 1.04 * | 0.93 | 1 * |
| (C18:0, C18:2)# | | | | |
| Threonic acid | 1.51 * | 1.13 * | 1.11 | 0.95 |

TABLE 1b
Markers for hematotoxicity in female rats; Significant
down-regulation changes (p-Value ≦ 0.2) are marked
(*). For some metabolites (marked with #), additional
information are provided in table 11. Bone marrow suppression
inducing Oxaliplatin was administered at low dose.

| | Carbo- | Cis- | Ibu- | |
|---|---|---|---|---|
| | platin | platin | profen | Oxaliplatin |
| Metabolite | f28 | f28 | f28 | f28 |
| Progesterone | 0.49* | 0.53 * | 0.41 * | 0.69 * |
| 4-Hydroxyphenylpyruvate | 0.39 * | 0.76 * | 0.84 * | 0.75 * |
| 21-Hydroxyprogesterone (11- | 0.09 * | 0.26 * | 0.19 * | 0.16 * |
| Deoxycorticosterone) | | | | |
| 18-Hydroxy-11- | 0.33 * | 0.46 * | 0.24 * | 0.37 * |
| deoxycorticosterone | | | | |
| Citrate | 0.78 * | 0.87 * | 0.94 * | 0.82 * |
| Corticosterone | 0.31 * | 0.49 * | 0.32 * | 0.43 * |

TABLE 1c
Markers for hematotoxicity in male rats; Significant up-regulation
changes (p-Value ≦ 0.2) are marked (*). For some metabolites
(marked with #), additional information are provided in table 11.

| | Cyclophosphamide | Adriamycin | |
|---|---|---|---|
| | monohydrate | hydrochloride | Cytarabin |
| Metabolite | m28 | m28 | m28 |
| Choline plasmalo- | 1.13 * | 1.18 * | 1.11 * |
| gen No 02# | | | |
| Valine | 1.25 * | 1.21 * | 1.1 * |
| Leucine | 1.33 * | 1.4 * | 1.12 * |
| Isoleucine | 1.23 * | 1.35 * | 1.1 * |
| Taurine | 1.21 * | 1.39 * | 1.27 * |
| Tricosanoic acid | 1.44 * | 2.14 * | 1.3 |
| (C23:0) | | | |
| Lignoceric acid | 1.29 * | 2.04 * | 1.31 * |
| (C24:0) | | | |

TABLE 1d
Markers for hematotoxicity in male rats; Significant down-regulation
changes (p-Value ≦ 0.2) are marked (*). For some metabolites
(marked with #), additional information are provided in table 11.

| | Cyclophosphamide | Adriamycin | |
|---|---|---|---|
| | monohydrate | hydrochloride | Cytarabin |
| Metabolite | m28 | m28 | m28 |
| Ketoleucine | 0.88 * | 0.69 * | 0.75 * |
| Citrate | 0.78 * | 0.86 | 0.81 * |
| 18-Hydroxy-11- | 0.16 * | 0.44 * | 0.13 * |
| deoxycorticosterone | | | |
| 3-Hydroxybutyrate | 0.75 * | 0.66 * | 0.53 * |

TABLE 1e
Markers for hematotoxicity in male rats; Significant up-regulation
changes (p-Value ≦ 0.2) are marked (*). For some metabolites
(marked with #), additional information are provided in table 11.

| | Cisplatin | Carboplatin |
|---|---|---|
| Metabolite | m28 | m28 |
| Tryptophan | 1.27 * | 1.17 * |
| Ornithine | 1.16 * | 1.11 * |
| Glucose-6-phosphate | 1.32 * | 2.2 * |
| Phenylalanine | 1.17 * | 1.2 * |
| Taurine | 1.29 * | 1.47 * |
| Indole-3-lactic acid | 1.27 * | 1.13 * |
| Fructose-6-phosphate | 1.05 * | 1.74 * |
| Aspartate | 1.18 * | 1.45 * |
| Histidine | 1.12 * | 1.17 * |
| Valine | 1.16 * | 1.29 * |
| Leucine | 1.36 * | 1.41 * |
| Isoleucine | 1.26 * | 1.3 * |
| Glycerol-3-phosphate, | 1.3 * | 2.21 * |
| polar fraction | | |

TABLE 1f
Markers for hematotoxicity in male rats; Significant down-regulation
changes (p-Value ≦ 0.2) are marked (*). For some metabolites
(marked with #), additional information are provided in table 11.

| | Cisplatin | Carboplatin |
|---|---|---|
| Metabolite | m28 | m28 |
| 14-Methylhexadecanoic | 0.83 * | 0.72 * |
| acid | | |
| 18-Hydroxy-11- | 0.42 * | 0.18 * |
| deoxycorticosterone | | |
| Choline plasmalogen | 0.89 * | 0.89 * |
| (C18, C20:4) | | |
| 3-Hydroxybutyrate | 0.73 * | 0.74 * |
| Citrate | 0.71 * | 0.76 * |
| Ascorbic acid | 0.83 * | 0.84 * |

TABLE 2a
Markers for hematotoxicity (blood anemia) in female rats; Significant up-regulation changes (p-Value ≦
0.15) are marked (*). For some metabolites (marked with #), additional information are provided in table 11.

| | 4-Chloro-3- | | | | | | 2- | | |
|---|---|---|---|---|---|---|---|---|---|
| | nitroaniline | | | Aniline | | | Chloroaniline | | |
| Metabolite | f7 | f14 | f28 | f7 | f14 | f28 | f7 | f14 | f28 |
| Ribal | 1.36 * | 1.36 * | 1.48 * | 1.67 * | 1.16 * | 1.7 * | 1.87 * | 1.26 * | 1.22 * |
| Cytosine | 1.57 * | 1.14 * | 1.54 * | 1.8 * | 1.3 * | 1.37 * | 1.82 * | 1.28 * | 1.22 * |
| TAG (C16:0, C18:2)# | 2.71 * | 2 * | 3.13 * | 2.24 * | 3.58 * | 4.71 * | 1.89 * | 1.67 * | 2.19 * |
| TAG (C18:2, C18:2)# | 3.38 * | 1.67 | 4.93 * | 1.97 * | 3.29 * | 5.17 * | 1.67 | 1.84 * | 2.12 * |
| TAG | 2.05 * | 1.92 | 3.93 * | 2.25 * | 3.59 * | 5.33 * | 1.9 * | 2.27 * | 1.74 * |
| (C16:0, C18:1, C18:3)# | | | | | | | | | |
| TAG No 05# | 1.79 | 2.34 * | 5.86 * | 2.17 * | 2.59 * | 4.41 * | 1.44 * | 1.33 * | 1.74 * |
| TAG (C18:1, C18:2)# | 3.13 * | 1.75 * | 3.36 * | 1.56 | 2.87 * | 3.77 * | 1.64 | 1.32 * | 1.59 * |
| TAG (DAG- | 3.64 * | 1.86 * | 3.77 * | 1.78 | 3.18 * | 5.12 * | 1.72 * | 1.39 * | 1.95 * |
| Fragment)# | | | | | | | | | |

TABLE 2b
Markers for hematotoxicity (blood anemia) in female rats; Significant down-
regulation changes (p-Value ≦ 0.15) are marked (*). For some metabolites
(marked with #), additional information are provided in table 11.

| | 4-Chloro-3- | | | | | | 2- | | |
|---|---|---|---|---|---|---|---|---|---|
| | nitroaniline | | | Aniline | | | Chloroaniline | | |
| Metabolite | f7 | f14 | f28 | f7 | f14 | f28 | f7 | f14 | f28 |
| 18-Hydroxy-11- | 0.25 * | 0.5 * | 0.75 * | 0.4 * | 0.63 * | 0.49 * | 0.7 * | 0.77 | 0.5 * |
| deoxycorticosterone | | | | | | | | | |
| TAG No 02# | 0.65 * | 0.85 * | 0.72 * | 0.67 * | 0.71 * | 0.55 * | 0.78 * | 0.85 * | 0.95 |
| Ketoleucine | 0.87 | 0.7 * | 0.88 * | 0.84 * | 0.83 | 0.86 * | 0.73 * | 1.07 | 0.79 * |
| Lactate | 0.83 | 0.43 * | 0.58 * | 0.98 | 0.7 | 0.73 * | 0.91 | 0.75 | 0.84 * |
| Pyruvate | 1.35 | 0.49 * | 0.45 * | 0.79 * | 0.84 | 0.59 * | 0.94 | 0.81 | 0.54 * |

TABLE 3a
Markers for hematotoxicity (Blood porhyrin inhibition) in female rats; Significant
up-regulation changes (p-Value ≦ 0.2) are marked (*). For some metabolites
(marked with #), additional information are provide in table 11.

| | | | | Lead acetate | | |
|---|---|---|---|---|---|---|
| | Saflufenacil | | | trihydrate | | |
| Metabolite | f7 | f14 | f28 | f7 | f14 | f28 |
| Urea | 1.35 * | 1.63 * | 1.06 | 1.22 * | 1.32 * | 1.13 * |
| TAG | 1.74 * | 2.17 * | 2 * | 1.42 * | 1.64 | 1.46 * |
| (C16:0, C18:1, C18:3)# | | | | | | |
| Phosphatidylcholine | 1.11 * | 1.08 | 1.2 * | 1.28 * | 1.19 | 1.22 * |
| (C16:0, C16:0)# | | | | | | |
| Creatinine | 1.47 * | 0.96 | 0.88 | 1.2 * | 1.09 | 1.02 |
| Lysophosphatidylcholine | 1.18 * | 1.04 | 1.19 | 1.21 * | 1.18 * | 0.91 |
| (C17:0)# | | | | | | |
| Phosphatidylcholine No | 0.98 | 1.16 * | 1.19 | 1.13 | 1.15 * | 1.23 |
| 02# | | | | | | |
| Aspartate | NA | 1.13 | 1.46 | 1.24 * | 1.06 | 1.3 * |
| DAG (C18:1, C18:2)# | 1.49 | 1.3 | 1.38 * | 1.5 * | 1.07 | 1.27 * |
| TAG No 07# | 1.43 * | 2.07 * | 1.89 * | 1.77 * | 1.33 | 0.97 |
| Ceramide (d18:1, C24:1) | 1.58 * | 0.77 | 1.39 | 1.07 | 1.07 * | 1.25 * |
| TAG No 05# | 1.29 * | 1.95 * | 2.03 * | 1.32 * | 1.53 | 1.38 * |
| Linoleic acid | 1.12 | 1.22 * | 1.27 * | 1.02 | 1.18 * | 1.1 |
| (C18:cis[9,12]2) | | | | | | |
| TAG (C16:0, C18:2)# | 1.59 * | 1.83 * | 1.77 * | 1.58 * | 1.31 | 1.41 * |
| TAG (C18:2, C18:3)# | 1.84 * | 1.49 | 1.27 * | 1.49 * | 1.48 | 1.3 |
| Glycerol, lipid fraction | 1.11 | 1.26 | 1.02 * | 1.23 * | 0.93 | 1.2 * |
| TAG (DAG-Fragment)# | 1.4 | 1.49 | 1.63 * | 1.71 * | 1.19 | 1.38 * |
| TAG No 059# | 1.57 * | 1.59 * | 1.4 * | 1.69 * | 1.17 | 1.25 * |

TABLE 3b
Markers for hematotoxicity (Blood porhyrin inhibition) in
female rats; Significant down-regulation changes (p-Value ≦
0.2) are marked (*). For some metabolites (marked with #),
additional information are provide in table 11.

| | | | | Lead acetate | | |
|---|---|---|---|---|---|---|
| | Saflufenacil | | | trihydrate | | |
| Metabolite | f7 | f14 | f28 | f7 | f14 | f28 |
| Valine | NA | 0.8 * | 0.71 * | 0.89 * | 0.91 | 0.76 * |
| Phenylalanine | 1.01 | 0.83 * | 0.78 * | 0.84 * | 0.94 * | 0.91 * |
| Histidine | NA | 0.83 * | 0.79 * | 0.92 | 0.99 | 0.77 * |
| TAG No 02# | 0.78 * | 0.78 * | 0.8 * | 0.95 * | 0.92 * | 0.88 |
| Ketoleucine | 0.98 | 0.76 * | 0.73 * | 1.04 | 0.84 * | 0.63 * |
| Lysine | NA | 0.73 * | 0.8 * | 1.15 | 1.05 | 0.82 * |

TABLE 3c
Markers for hematotoxicity (Blood porhyrin inhibition) in
female rats; Significant up-regulation changes (p-Value ≦
0.2) are marked (*). For some metabolites (marked with #),
additional information are provide in table 11.

| | | | Lead |
|---|---|---|---|
| | | | acetate |
| | | Saflufenacil | trihydrate |
| Metabolite | Direction | f28 | f28 |
| Sphingomyelin | up | 1.11 * | 1.09 * |
| (d18:2, C18:0)# | | | |
| DAG (C18:1, C18:2)# | up | 1.38 * | 1.27 * |
| Phosphatidylcholine | up | 1.2 * | 1.22 * |
| (C16:0, C16:0)# | | | |
| TAG No 05# | up | 2.03 * | 1.38 * |
| TAG (C16:0, C18:2)# | up | 1.77 * | 1.41 * |
| TAG | up | 2 * | 1.46 * |
| (C16:0, C18:1, C18:3)# | | | |
| Glycerol, lipid fraction | up | 1.02 * | 1.2 * |
| Cholesterolester | up | 1.12 * | 1.28 * |
| No 01# | | | |
| Phosphatidylcholine | up | 1.01 * | 1.06 * |
| (C16:0, C18:2)# | | | |
| TAG (C18:2, C18:2)# | up | 1.67 * | 1.48 * |
| TAG No 01# | up | 1.29 * | 1.5 * |
| TAG (C18:1, C18:2)# | up | 1.3 * | 1.33 * |
| TAG (DAG-Fragment)# | up | 1.63 * | 1.38 * |
| TAG No 059# | up | 1.4 * | 1.25 * |

TABLE 3d
Markers for hematotoxicity (Blood porhyrin inhibition) in
female rats; Significant down-regulation changes (p-Value ≦
0.2) are marked (*). For some metabolites (marked with #),
additional information are provide in table 11.

| | | | Lead |
|---|---|---|---|
| | | | acetate |
| | | Saflufenacil | trihydrate |
| Metabolite | Direction | f28 | f28 |
| Lysine | down | 0.8 * | 0.82 * |
| Malate | down | 0.77 * | 0.73 * |
| Isoleucine | down | 0.74 * | 0.72 * |
| Valine | down | 0.71 * | 0.76 * |
| Ketoleucine | down | 0.73 * | 0.63 * |
| Leucine | down | 0.76 * | 0.72 * |
| Phenylalanine | down | 0.78 * | 0.91 * |
| Proline | down | 0.85 * | 0.91 * |
| Methionine | down | 0.84 * | 0.89 * |
| Alanine | down | 0.8 * | 0.81 * |
| 18-Hydroxy-11- | down | 0.57 * | 0.4 * |
| deoxycorticosterone | | | |

TABLE 3e
Markers for hematotoxicity (Blood porhyrin inhibition) in
male rats; Significant up-regulation changes (p-Value ≦
0.2) are marked (*). For some metabolites (marked with #),
additional information are provide in table 11.

| | | | | Lead acetate | | |
|---|---|---|---|---|---|---|
| | Saflufenacil | | | trihydrate | | |
| Metabolite | m7 | m14 | m28 | m7 | m14 | m28 |
| Choline | 1.21 * | 1.21 * | 1.23 * | 1.11 * | 1.05 | 0.94 |
| plasmalogen | | | | | | |
| No 03# | | | | | | |
| Choline | 1.1 * | 1.42 * | 1.15 * | 1.05 | 1.16 * | 0.93 |
| plasmalogen | | | | | | |
| No 02# | | | | | | |

TABLE 3f
Markers for hematotoxicity (Blood porhyrin inhibition) in
male rats; Significant down-regulation changes (p-Value ≦
0.2) are marked (*). For some metabolites (marked with #),
additional information are provide in table 11.

| | | | | Lead acetate | | |
|---|---|---|---|---|---|---|
| | Saflufenacil | | | trihydrate | | |
| Metabolite | m7 | m14 | m28 | m7 | m14 | m28 |
| Isoleucine | 0.87 * | 0.89 * | 0.89 * | 0.86 * | 0.94 * | 0.87 * |
| Methionine | 0.86 * | 0.86 * | 0.86 * | 0.95 * | 0.96 * | 0.89 * |
| Leucine | 0.84 * | 0.82 * | 0.8 * | 0.87 * | 0.91 * | 0.82 * |
| Serine | 0.86 * | 0.92 * | 0.98 * | 0.85 * | 0.85 * | 0.9 * |
| Threonic acid | 0.5 * | 0.62 * | 0.61 * | 0.67 * | 0.68 * | 0.77 |
| Valine | 0.82 * | 0.85 * | 0.87 * | 0.88 * | 0.93 * | 0.87 * |
| Alanine | 0.68 * | 0.83 * | 0.83 * | 0.68 * | 0.8 * | 0.77 * |
| Uric acid | 0.65 * | 0.67 * | 0.83 * | 0.64 * | 0.86 | 0.76 * |
| Phenylalanine | 0.91 * | 0.91 * | 0.8 * | 0.83 * | 0.89 * | 0.87 * |
| 5-Oxoproline | 0.84 * | 0.84 * | 0.74 * | 0.88 * | 0.93 * | 0.93 |
| Glutamine | 0.81 * | 0.84 * | 0.79 * | 0.83 * | 0.85 * | 0.92 * |
| Ketoleucine | 0.74 * | 0.76 * | 0.86 * | 0.66 * | 0.89 * | 0.73 * |
| Malate | 0.36 * | 0.46 * | 0.42 * | 0.69 * | 1.08 | 0.75 * |
| 18-Hydroxy- | 0.47 * | 0.49 * | 0.3 * | 0.73 | 0.46 * | 0.67 |
| 11-deoxycorti- | | | | | | |
| costerone | | | | | | |
| trans-4- | 0.93 * | 0.98 | 0.92 * | 0.81 | 0.82 * | 0.97 |
| Hydroxyproline | | | | | | |
| myo-Inositol | 0.77 * | 1.06 | 0.77 * | 0.72 * | 0.93 | 1.16 |

TABLE 3g
Markers for hematotoxicity (Blood porhyrin inhibition) in
male rats; Significant down-regulation changes (p-Value ≦
0.2) are marked (*). For some metabolites (marked with #),
additional information are provide in table 11.

| | | Lead |
|---|---|---|
| | | acetate |
| | Saflufenacil | trihydrate |
| Metabolite | m28 | m28 |
| Isoleucine | 0.89 * | 0.87 * |
| Methionine | 0.86 * | 0.89 * |
| Phenylalanine | 0.8 * | 0.87 * |
| Leucine | 0.8 * | 0.82 * |
| Valine | 0.87 * | 0.87 * |
| Serine | 0.98 * | 0.9 * |
| Glucose | 0.82 * | 0.91 |
| Uric acid | 0.83 * | 0.76 * |
| Ketoleucine | 0.86 * | 0.73 * |
| Alanine | 0.83 * | 0.77 * |
| Glutamine | 0.79 * | 0.92 * |
| Malate | 0.42 * | 0.75 * |
| 5-Oxoproline | 0.74 * | 0.93 |
| Proline | 0.87 * | 0.82 * |

TABLE 4a
Markers for hematotoxicity (spleen methemoglobin) in female rats; Significant up-regulation changes (p-Value ≦
0.1) are marked (*). For some metabolites (marked with #), additional information are provide in table 11.

| | 4-Chloro-3- | | | Lithocholic | | | 1,3- | | 1,4- | | |
|---|---|---|---|---|---|---|---|---|---|---|---|
| | nitroaniline | | | acid | | | Dinitrobenzene | | Dinitrobenzene | | |
| Metabolite | f7 | f14 | f28 | f7 | f14 | f28 | f7 | f14 | f7 | f14 | f28 |
| Serine | 1.18 | 1.43 * | 1.42 * | 1.2 * | 1.29 * | 1.97 * | 1.22 * | 1.14 * | 1.23 * | 1.35 * | 1.27 * |
| Ribal | 1.88 * | 1.58 * | 1.52 * | 1.17 * | 1.05 | 1.19 * | 1.69 * | 1.34 * | 2.42 * | 1.55 * | 2.3 * |
| Cytosine | 1.69 * | 1.38 * | 1.49 * | 1.23 * | 1.1 | 1.26 * | 1.75 * | 1.44 * | 2.57 * | 1.66 * | 2.1 * |
| Threonine | 1.55 * | 1.95 * | 2.05 * | 1.26 * | 1.25 * | 2.4 * | 1.59 * | 1.22 * | 1.47 * | 1.24 * | 1.67 * |
| Docosahexaenoic acid | 1.31 * | 1.71 * | 1.43 * | 1.09 * | 1.05 | 1.39 | 1.26 | 1.58 * | 1.28 * | 1.29 * | 1.22 * |
| (C22:cis[4,7,10,13,16,19]6) | | | | | | | | | | | |
| 3-Methoxytyrosine | NA | NA | NA | 1.32 * | 1.22 * | 1.36 * | 1.61 * | 1.2 * | 1.46 * | 1.36 * | 1.47 * |
| Phytosphingosine | 1.45 * | 1.66 * | 1.81 * | 1.34 * | 1.1 | 1.44 * | 1.31 * | 1.49 * | 1.27 * | 1.08 * | 1.34 * |
| Linoleic acid | 1.26 | 1.58 * | 1.59 * | 1.61 * | 1.29 * | 2 * | 1.34 | 1.37 * | 1.44 * | 1.67 * | 1.72 * |
| (C18:cis[9,12]2) | | | | | | | | | | | |
| Threonic acid | 1.25 * | 1.45 * | 1.26 * | 1.35 | 1.24 * | 1.45 * | 0.92 | 1.22 * | 1.3 * | 1.67 * | 1.28 * |

TABLE 4b
Markers for hematotoxicity (spleen methemoglobin) in female rats; Significant down-regulation changes (p-Value ≦
0.1) are marked (*). For some metabolites (marked with #), additional information are provide in table 11.

| | 4-Chloro-3- | | | Lithocholic | | | 1,3- | | 1,4- | | |
|---|---|---|---|---|---|---|---|---|---|---|---|
| | nitroaniline | | | acid | | | Dinitrobenzene | | Dinitrobenzene | | |
| Metabolite | f7 | f14 | f28 | f7 | f14 | f28 | f7 | f14 | f7 | f14 | f28 |
| Ketoleucine | 0.55 * | 0.44 * | 0.65 * | 1.03 | 0.94 | 0.88 * | 0.83 | 0.78 * | 0.81 * | 0.72 * | 0.71 * |
| Lactate | 1.04 | 0.51 * | 0.61 * | 0.96 | 0.62 * | 0.86 * | 0.69 * | 0.65 * | 1.09 | 0.79 * | 0.66 * |

TABLE 4c
Markers for hematotoxicity (spleen methemoglobin) in male rats; Significant up-regulation changes (p-Value ≦
0.1) are marked (*). For some metabolites (marked with #), additional information are provide in table 11.

| | 4-Chloro-3- | | | | | | 1,3- | | 1,4- | | |
|---|---|---|---|---|---|---|---|---|---|---|---|
| | nitroaniline | | | Flutamide | | | Dinitrobenzene | | Dinitrobenzene | | |
| Metabolite | m7 | m14 | m28 | m7 | m14 | m28 | m7 | m14 | m7 | m14 | m28 |
| Threonine | 1.46 * | 1.58 * | 1.68 * | 1.33 * | 1.24 * | 1.22 * | 1.32 * | 1.39 | 1.58 * | 1.37 * | 1.32 * |
| Serine | 1.57 * | 1.77 * | 1.48 * | 1.47 * | 1.52 * | 1.2 * | 1.22 * | 1.38 * | 1.39 * | 1.39 * | 1.39 * |
| Glycine | 1.4 * | 1.42 * | 1.34 * | 1.22 | 1.25 * | 1.17 * | 1.19 * | 1.13 * | 1.34 * | 1.24 * | 1.2 * |
| Serotonin (5- | 9.5 * | 3.45 * | 4.88 * | 2.29 * | 1.88 * | 1.56 * | 1.8 | 2.92 * | 3.51 * | 4.53 * | 2.57 * |
| HT) | | | | | | | | | | | |
| 3- | 1.9 * | 2.05 * | 2.23 * | 1.97 * | 1.47 * | 1.55 * | 1.43 * | 1.8 * | 1.75 * | 1.67 * | 1.52 * |
| Methoxytyrosine | | | | | | | | | | | |
| Proline | 1.28 * | 1.43 * | 1.41 * | 1.18 * | 1.37 * | 1.13 | 1.05 | 1.1 * | 1.28 * | 1.29 * | 1.27 * |
| Eicosanoic acid | 1.32 * | 1.55 * | 1.75 * | 2.11 * | 1.84 * | 1.93 | 1.23 * | 1.44 * | 1.71 * | 1.98 * | 2.14 * |
| (C20:0) | | | | | | | | | | | |
| Ribal | 1.89 * | 1.93 * | 1.74 * | 0.62 * | 0.76 | 0.86 | 1.43 * | 1.29 * | 1.61 * | 1.43 * | 1.98 * |

TABLE 4d
Markers for hematotoxicity (spleen methemoglobin) in male rats; Significant down-regulation changes (p-Value ≦
0.1) are marked (*). For some metabolites (marked with #), additional information are provide in table 11.

| | 4-Chloro-3- | | | | | | 1,3- | | 1,4- | | |
|---|---|---|---|---|---|---|---|---|---|---|---|
| | nitroaniline | | | Flutamide | | | Dinitrobenzene | | Dinitrobenzene | | |
| Metabolite | m7 | m14 | m28 | m7 | m14 | m28 | m7 | m14 | m7 | m14 | m28 |
| Urea | 0.89 | 0.7 * | 0.89 | 0.82 * | 0.82 | 0.73 | 0.79 * | 0.63 * | 0.81 * | 0.81 * | 0.82 * |
| Palmitoleic acid | 0.63 * | 0.51 * | 0.49 * | 0.77 * | 0.68 * | 0.6 * | 0.42 * | 0.22 * | 0.92 | 0.55 * | 0.38 * |
| (C16:cis[9]1) | | | | | | | | | | | |
| 3,4- | 0.23 * | 0.16 * | 0.19 * | 0.6 * | 0.67 * | 0.47 * | 0.71 * | 0.67 * | 0.84 * | 0.94 | 0.54 * |
| Dihydroxyphenylacetic | | | | | | | | | | | |
| acid (DOPAC) | | | | | | | | | | | |
| Elaidic acid | 0.77 * | 0.77 * | 0.79 * | 0.97 | 0.8 * | 0.6 * | 0.6 * | 0.7 * | 0.83 * | 0.68 | 0.58 * |
| (C18:trans[9]1) | | | | | | | | | | | |

TABLE 5a
Markers for hematotoxicity (spleen heamosiderosis) in female rats; Significant up-regulation changes (p-Value ≦
0.2) are marked (*). For some metabolites (marked with #), additional information are provide in table 11.

| | Cyclohexanone | | | 1,4- | | | 4-Chloro-3- | | |
|---|---|---|---|---|---|---|---|---|---|
| | oxime (CHO) | | | Dinitrobenzene | | | nitroaniline | | |
| Metabolite | f7 | f14 | f28 | f7 | f14 | f28 | f7 | f14 | f28 |
| Linoleic acid | 1.46 * | 1.21 * | 1.13 * | 1.44 * | 1.67 * | 1.72 * | 1.26 | 1.58 * | 1.59 * |
| (C18:cis[9,12]2) | | | | | | | | | |
| Docosahexaenoic acid | 1.72 * | 1.55 * | 1.6 * | 1.28 * | 1.29 * | 1.22 * | 1.31 * | 1.71 * | 1.43 * |
| (C22:cis[4,7,10,13,16,19]6) | | | | | | | | | |
| Heptadecanoic acid | 1.14 * | 1.34 * | 1.32 * | 1.15 | 1.19 * | 1.24 * | 1.07 | 1.42 * | 1.38 * |
| (C17:0) | | | | | | | | | |
| Phytosphingosine | 1.33 * | 1.27 * | 1.31 * | 1.27 * | 1.08 * | 1.34 * | 1.45 * | 1.66 * | 1.81 * |
| Cytosine | 1.31 * | 1.13 * | 1.45 * | 2.57 * | 1.66 * | 2.1 * | 1.69 * | 1.38 * | 1.49 * |
| Ribal | 1.15 * | 1.16 | 1.38 * | 2.42 * | 1.55 * | 2.3 * | 1.88 * | 1.58 * | 1.52 * |
| 3-Methoxytyrosine | NA | NA | NA | 1.46 * | 1.36 * | 1.47 * | NA | NA | NA |
| Palmitic acid (C16:0) | 1.45 * | 1.28 * | 1.22 * | 1.19 * | 1.24 * | 1.42 * | 1.12 | 1.34 * | 1.34 * |
| Threonine | 1.22 * | 1.53 * | 1.47 * | 1.47 * | 1.24 * | 1.67 * | 1.55 * | 1.95 * | 2.05 * |
| 3-Hydroxyindole | 1.59 * | 1.55 * | 0.75 | 1.55 * | 1.79 * | 1.63 * | 1.76 * | 1.97 * | 2.79 * |
| Serotonin (5-HT) | NA | NA | NA | 1.46 | 2.66 * | 3.09 * | NA | NA | NA |
| Glycerol, lipid fraction# | 1.12 * | 1.11 * | 1.49 * | 1.41 * | 1.86 * | 2.17 * | 1.07 | 1.45 * | 1.56 * |
| Phosphate (inorganic and | 1.24 * | 1.31 * | 1.09 | 1.11 | 1.2 * | 1.22 * | 0.92 * | 1.11 * | 1.21 * |
| organic phosphates) | | | | | | | | | |
| Androstenedione | NA | NA | NA | 0.9 | 1.97 * | 2.34 * | NA | NA | NA |
| Glucuronic acid | 0.96 | 1.2 * | 1.22 * | 1.97 * | 1.86 * | 1.66 * | 2.62 * | 2.29 * | 1.52 * |
| Glutamate | 1.12 | 1.15 * | 1.27 * | 1.36 * | 1.52 * | 1.22 * | 1.13 * | 1.3 * | 1.46 * |

TABLE 5b
Markers for hematotoxicity (spleen heamosiderosis) in female rats; Significant
down-regulation changes (p-Value ≦ 0.2) are marked (*). For some metabolites
(marked with #), additional information are provide in table 11.

| | Cyclohexanone | | | 1,4- | | | 4-Chloro-3- | | |
|---|---|---|---|---|---|---|---|---|---|
| | oxime (CHO) | | | Dinitrobenzene | | | nitroaniline | | |
| Metabolite | f7 | f14 | f28 | f7 | f14 | f28 | f7 | f14 | f28 |
| Homovanillic acid | NA | NA | NA | 1.23 | 0.79 * | 0.52 * | NA | NA | NA |
| (HVA) | | | | | | | | | |
| Pyruvate | 0.78 * | 0.59 * | 0.66 * | 0.83 * | 0.59 * | 0.55 * | 0.98 | 0.53 * | 0.51 * |
| Ketoleucine | 1.02 | 0.92 * | 0.74 * | 0.81 * | 0.72 * | 0.71 * | 0.55 * | 0.44 * | 0.65 * |
| 3,4- | NA | NA | NA | 0.94 * | 0.72 * | 0.7 * | NA | NA | NA |
| Dihydroxyphenylglycol | | | | | | | | | |
| (DOPEG) | | | | | | | | | |
| Progesterone | NA | NA | NA | 0.84 * | 0.41 * | 0.74 * | NA | NA | NA |

TABLE 5c
Markers for hematotoxicity (spleen heamosiderosis) in male rats; Significant up-regulation changes (p-Value ≦
0.2) are marked (*). For some metabolites (marked with #), additional information are provide in table 11.

| | Cyclohexanone | | | | | | 1,4- | | | 4-Chloro-3- | | |
|---|---|---|---|---|---|---|---|---|---|---|---|---|
| | oxime (CHO) | | | Linuron | | | Dinitrobenzene | | | nitroaniline | | |
| Metabolite | m7 | m14 | m28 | m7 | m14 | m28 | m7 | m14 | m28 | m7 | m14 | m28 |
| Ribal | 1.47 * | 1.56 * | 1.77 * | 1.46 * | 1.36 * | 1.26 * | 1.61 * | 1.43 * | 1.98 * | 1.51 * | 1.57 * | 1.52 * |
| Docosahexaenoic acid | 1.41 * | 1.36 * | 1.35 * | 1.05 | 1.25 | 1.22 * | 1.58 * | 1.55 * | 1.55 * | 1.33 | 1.36 * | 1.22 |
| (C22:cis[4,7,10,13,16,19]6) | | | | | | | | | | | | |
| Cytosine | 1.39 * | 1.49 * | 1.68 * | 1.13 * | 1.31 * | 1.05 | 1.66 * | 1.37 * | 1.97 * | 1.31 * | 1.64 * | 1.71 * |
| Threonic acid | 1.21 * | 1.34 * | 1.34 * | 1.9 * | 1.77 * | 1.24 * | 1.04 | 1.31 * | 1.19 * | 0.85 | 1.15 | 1.24 * |
| Pantothenic acid | 1.25 | 1.37 * | 1.68 * | 1.54 * | 1.96 * | 2.24 * | 1.06 | 1.24 * | 1.12 * | 1.27 * | 1.51 * | 0.98 |
| Xylitol | 1.59 * | 1.55 * | 2.08 * | 2.86 * | 2.93 * | 2.06 * | 1.51 * | 1.26 | 1.99 * | 1.7 * | 1.45 * | 1.44 |
| Linoleic acid | 1.31 * | 1.18 * | 1.18 | 1.44 * | 1.49 * | 1.5 * | 1.68 * | 1.88 * | 1.56 * | 1.1 * | 1.24 * | 1.39 * |
| (C18:cis[9,12]2) | | | | | | | | | | | | |
| Glucuronic acid | 1.35 * | 1.36 * | 1.74 * | 4.64 * | 2.54 * | 2.25 * | 1.27 * | 1.09 | 1.18 * | 2 * | 1.68 * | 1.08 |
| Phytosphingosine | 1.18 * | 1.15 | 0.94 | 1.89 * | 2.14 * | 2.47 * | 1.28 * | 1.07 | 1.12 * | 1.93 * | 1.6 * | 1.3 |
| Threonine | 1.25 * | 1.23 * | 1.28 * | 1.48 * | 1.62 * | 1.53 * | 1.5 * | 1.32 * | 1.36 * | 1.37 * | 1.4 * | 1.47 * |
| Allantoin | 1.1 * | 1.25 * | 1.35 * | 0.98 | 1.36 * | 1.1 * | 1.2 * | 1.19 * | 1.42 * | 1.43 * | 1.2 * | 1.21 * |

TABLE 5d
Markers for hematotoxicity (spleen heamosiderosis) in male rats; Significant down-regulation changes (p-Value ≦
0.2) are marked (*). For some metabolites (marked with #), additional information are provide in table 11.

| | Cyclohexanone | | | | | | 1,4- | | | 4-Chloro-3- | | |
|---|---|---|---|---|---|---|---|---|---|---|---|---|
| | oxime (CHO) | | | Linuron | | | Dinitrobenzene | | | nitroaniline | | |
| Metabolite | m7 | m14 | m28 | m7 | m14 | m28 | m7 | m14 | m28 | m7 | m14 | m28 |
| Palmitoleic acid | 1.22 | 0.78 | 0.8 * | 0.75 * | 0.5 * | 0.77 * | 0.92 | 0.55 * | 0.38 * | 0.64 * | 0.59 * | 0.44 * |
| (C16:cis[9]1) | | | | | | | | | | | | |
| 16- | 1.14 | 0.82 * | 0.72 * | 0.75 * | 0.69 * | 0.69 * | 0.91 | 1.01 | 0.61 * | 0.88 * | 0.84 * | 1.05 |
| Methylheptadecanoic | | | | | | | | | | | | |
| acid | | | | | | | | | | | | |

TABLE 6a
Markers for hematotoxicity (systemic anti-proliferation) in
female rats; Significant up-regulation changes (p-Value ≦
0.1) are marked (*). For some metabolites (marked
with #), additional information are provide in table 11.
Oxaliplatin was administered in high dose.

| | | | | Adriamycin | | |
|---|---|---|---|---|---|---|
| | Oxaliplatin | | | hydrochloride | | |
| Metabolite | f7 | f14 | f28 | f7 | f14 | f28 |
| Coenzyme Q9 | 1.18 * | 1.48 * | 1.63 * | 1.98 * | 1.71 * | 2.3 * |
| Coenzyme | 1.31 * | 1.67 * | 1.56 * | 1.22 | 1.19 * | 1.82 * |
| Q10 | | | | | | |
| Cytosine | 1.27 * | 1.5 * | 1.34 * | 1.77 * | 1.71 * | 2.05 * |
| Mannose | 1.13 | 1.24 * | 1.23 * | 1.35 * | 1.31 * | 1.36 * |
| Ribal | 1.48 * | 1.41 | 1.47 * | 1.51 * | 1.6 * | 2.11 * |
| Leucine | 1.23 * | 1.13 * | 1.43 * | 1 | 1.24 * | 1.36 * |
| Isoleucine | 1.27 * | 1.11 * | 1.36 * | 0.97 | 1.21 * | 1.28 * |
| Valine | 1.21 * | 1.08 * | 1.31 * | 0.95 | 1.14 * | 1.27 * |

TABLE 6b
Markers for hematotoxicity (systemic anti-proliferation) in female
rats; Significant down-regulation changes (p-Value ≦ 0.1) are
marked (*). For some metabolites (marked with #), additional information
are provide in table 11. Oxaliplatin was administered in high dose.

| | | | | Adriamycin | | |
|---|---|---|---|---|---|---|
| | Oxaliplatin | | | hydrochloride | | |
| Metabolite | f7 | f14 | f28 | f7 | f14 | f28 |
| Creatinine | 0.69 * | 0.77 * | 0.81 * | 0.77 * | 0.95 * | 0.92 |
| Malate | 0.85 * | 0.67 * | 1.3 | 0.55 * | 0.67 * | 0.93 |

TABLE 7a
Markers for hematotoxicity (blood aplastic anemia) in male rats; Significant
up-regulation changes (p-Value ≦ 0.1) are marked (*). For some metabolites
(marked with #), additional information are provide in table 11.

| | Probenecid | | | Methimazole | | |
|---|---|---|---|---|---|---|
| Metabolite | m7 | m14 | m28 | m7 | m14 | m28 |
| gamma-Linolenic acid | 2.14 * | 2.74 * | 2.26 * | 1.85 * | 2.63 * | 2.23 * |
| (C18:cis[6,9,12]3) | | | | | | |
| Sphingomyelin | 1.39 * | 1.49 * | 1.23 * | 1.35 * | 1.64 * | 1.41 * |
| (d18:1, C24:0)# | | | | | | |
| Histidine | 1.31 * | 1.4 * | 1.34 * | 1.25 * | 1.2 * | 1.21 * |
| Choline plasmalogen | 1.36 * | 1.19 * | 1.38 * | 1.24 * | 1.34 * | 1.21 * |
| No 01# | | | | | | |
| Ornithine | 1.37 * | 1.09 | 1.3 * | 1.17 * | 1.34 * | 1.59 * |
| Coenzyme Q9 | 1.66 * | 1.63 * | 1.02 | 1.23 | 1.54 * | 2.14 * |
| Phosphatidylcholine | 1.2 * | 1.31 * | 1.28 * | 1.36 * | 1.55 * | 1.6 * |
| (C18:0, C18:1)# | | | | | | |
| Phosphatidylcholine | 1.08 * | 1.12 * | 1.13 * | 1.11 * | 1.28 * | 1.35 * |
| (C18:0, C18:2)# | | | | | | |
| Phosphatidylcholine | 1.28 * | 1.36 * | 1.15 * | 1.05 * | 1.35 * | 1.35 * |
| (C16:0, C20:5)# | | | | | | |
| Leucine | 1.18 * | 1.1 | 1.18 * | 1.24 * | 1.34 * | 1.78 * |
| Phytosphingosine | 1.44 * | 2.24 * | 1.96 * | 2 * | 3.12 * | 3.11 * |
| dihomo-gamma- | 1.48 * | 1.43 * | 1.41 * | 2.38 * | 3.18 * | 2.09 * |
| Linolenic acid | | | | | | |
| (C20:cis[8,11,14]3) | | | | | | |
| Eicosanoic acid | 1.39 | 1.29 * | 1.18 | 1.32 * | 1.71 * | 1.74 * |
| (C20:0) | | | | | | |
| Valine | 1.13 * | 1.2 * | 1.09 * | 1.28 * | 1.5 * | 1.77 * |
| alpha-Tocopherol | 1.16 | 1.31 * | 1.11 * | 2.26 * | 3.65 * | 2.04 * |

TABLE 7b
Markers for hematotoxicity (blood aplastic anemia) in
male rats; Significant down-regulation changes (p-Value ≦
0.1) are marked (*). For some metabolites (marked with #),
additional information are provide in table 11.

| | Probenecid | | Methimazole | | | |
|---|---|---|---|---|---|---|
| Metabolite | m7 | m14 | m28 | m7 | m14 | m28 |
| Cytosine | 0.7 * | 0.69 * | 0.68 * | 0.64 * | 0.44 * | 0.32 * |
| Phosphatidyl- | 0.53 * | 0.79 * | 0.68 * | 0.84 * | 0.75 * | 0.64 * |
| choline No 04# | | | | | | |
| Ribal | 0.78 * | 0.87 * | 0.68 * | 0.65 * | 0.46 * | 0.42 * |
| 16- | 0.63 * | 0.64 * | 0.55 * | 1.21 * | 0.73 * | 0.47 * |
| Methylhepta- | | | | | | |
| decanoic acid | | | | | | |

TABLE 8a
Markers for hematotoxicity (Immunosupression) in male
rats; Significant up-regulation changes (p-Value ≦
0.2) are marked (*). For some metabolites (marked with
#), additional information are provide in table 11.

| | Cyclosporin | | |
|---|---|---|---|
| | A | Tacrolimus | Methylprednisolone |
| Metabolite | m28 | m28 | m28 |
| Cholesterolester | 1.15 * | 1.05 * | 1.08 * |
| No 01# | | | |
| Glutamate | 1.16 * | 1.29 * | 1.11 * |
| Aspartate | 1.31 * | 1.42 * | 1.37 * |
| Glycerol-3-phosphate, | 1.2 * | 1.54 * | 1.4 * |
| polar fraction# | | | |

TABLE 8b
Markers for hematotoxicity (Immunosupression) in male
rats; Significant down-regulation changes (p-Value ≦
0.2) are marked (*). For some metabolites (marked with
#), additional information are provide in table 11.

| | Cyclosporin | | |
|---|---|---|---|
| | A | Tacrolimus | Methylprednisolone |
| Metabolite | m28 | m28 | m28 |
| Ketoleucine | 0.65 * | 0.85 * | 0.75 * |
| 18-Hydroxy-11- | 0.23 * | 0.48 * | 0.15 * |
| deoxycorticosterone | | | |
| Pyruvate | 0.89 * | 0.77 * | 0.65 * |

TABLE 9
Markers for hematotoxicity (spleen haematopiesis) in male rats; Significant
down-regulation changes (p-Value ≦ 0.2) are marked (*). For some metabolites
(marked with #), additional information are provided in table 11.

| | Epoxiconazole | | | Triethanolamine | | | 2-Butoxyethanol | | |
|---|---|---|---|---|---|---|---|---|---|
| Metabolite | m7 | m14 | m28 | m7 | m14 | m28 | m7 | m14 | m28 |
| Uric acid | 0.85 * | 0.69 * | 0.62 * | 0.76 * | 0.52 * | 0.67 * | 0.75 * | 0.71 * | 0.66 * |
| Cytosine | 0.82 * | 0.78 * | 0.84 * | 0.91 | 0.86 * | 0.92 * | 0.86 * | 0.93 | 0.86 * |
| Uracil | 0.91 | 0.84 * | 0.93 | 0.79 * | 0.81 * | 1.07 | 0.77 * | 0.9 | 0.71 * |
| Ascorbic acid | 0.9 * | 0.9 * | 0.89 * | 1.04 | 0.85 * | 0.83 * | 0.91 | 0.8 * | 0.94 * |
| Ribal | 0.77 * | 0.8 * | 0.78 * | 0.82 * | 0.7 * | 0.86 * | 0.82 * | 1.06 | 0.91 * |
| Glucose | 0.72 * | 0.79 * | 0.71 * | 1.13 * | 0.82 * | 0.82 * | 0.81 * | 0.73 * | 0.93 * |
| 18-Hydroxy-11- | 0.31 * | 0.4 * | 0.93 | 0.36 * | 0.17 * | 0.29 | 0.18 * | 0.44 * | 0.68 * |
| deoxycorticosterone | | | | | | | | | |
| Malate | 1.04 | 0.66 * | 0.62 * | 0.83 | 0.61 * | 0.75 * | 0.81 * | 0.79 * | 0.7 * |
| Alanine | 0.87 * | 0.74 * | 0.85 * | 0.78 * | 0.7 * | 0.92 * | 0.83 * | 0.93 | 0.91 * |
| myo-Inositol | 0.85 * | 1.02 | 1.21 * | 0.7 * | 0.7 * | 0.82 * | 0.88 | 0.82 * | 0.93 * |
| Phenylalanine | 0.94 * | 0.92 * | 0.98 | 0.85 * | 0.91 * | 0.9 | 0.87 * | 0.94 * | 0.95 * |
| Lysophosphatidylcholine | 0.76 * | 0.67 * | 0.7 * | 0.85 * | 0.89 * | 0.78 * | 0.89 * | 0.85 * | 0.94 |
| (C20:4)# | | | | | | | | | |

TABLE 10
Compounds and dosing (CMC = Carboxymethyl cellulose)

| Compound | Synonym | CAS no | Dosage administered | Details |
|---|---|---|---|---|
| 1,3-Dinitrobenzene | m-Dinitrobenzene | 99-65-0 | 10 mg/kg body weight | in corn oil; administra- |
| | | | by gavage | tion volume: 5 ml/kg |
| | | | | bw |
| 1,4-Dinitrobenzene | p-Dinitrobenzene | 100-25-4 | 20 mg/kg body weight | in corn oli, administra- |
| | | | by gavage | tion volume: 5 ml/kg |
| | | | | body weight |
| 2-Butoxyethanol | na | 111-76-2 | 6,000 ppm in the diet | mixture in the diet |
| 2-Chloroaniline | 1-Amino-2-chlorobenzene | 95-51-2 | 160 mg/kg body weight | in corn oil; administra- |
| | | | by gavage | tion volume: 5 ml/kg |
| | | | | body weight |
| 4-Chloro-3-nitroaniline | 4-chloro-3-nitro benzenamine | 635-22-3 | 90 mg/kg body weight | in corn oil; administra- |
| | | | by gavage | tion volume: 5 ml/kg |
| | | | | body weigh |
| Adriamycin hydrochloride | Doxorubicin | 25316-40-9 | 2 mg/kg bw subcutaneous, | in 0.9% NaCl; admin- |
| | | | once weekly | istration volume: 1 |
| | | | | ml/kg bw; once weekly |
| | | | | on days 0, 6, 13, 20 |
| | | | | and 27 |
| Aniline | Aminobenzene | 62-53-3 | 100 mg/kg body weight | solution in aqua |
| | | | by gavage | bidest; administration |
| | | | | volume: 10 ml/kg body |
| | | | | weight |
| Saflufenacil | N′-[2-Chlor-4-fluor-5-(3-methyl-2,6- | 372137-35-4 | 1,000 ppm in the diet | mixture in the diet |
| | dioxo-4-(trifluormethyl)-3,6-dihydro- | | | |
| | 1(2H)-pyrimidinyl)benzoyl]-N- | | | |
| | isopropyl-N-methylsulfamid | | | |
| Cyclohexanone oxime (CHO) | (hydroxyimino)cyclo-hexane | 100-64-1 | 200 mg/kg body weight | in aqua bidest., ad- |
| | | | by gavage | ministration volume: |
| | | | | 15 ml/kg body weight |
| Epoxiconazole | na | 106325-08-0 | 2,000 ppm in the diet | mixture in the diet |
| Flutamide | na | 13311-84-7 | 100 mg/kg body weight | in corn oil, administra- |
| | | | by gavage | tion volume: 5 ml/kg |
| | | | | body weight |
| Lead acetate trihydrate | na | 6080-56-4 | 500 ppm in the diet | mixture in the diet |
| Linuron | 3-(3,4-dichlorophenyl)-1-methoxy-1- | 330-55-2 | 100 mg/kg body weight | in corn oil, administra- |
| | methylurea | | by gavage | tion volume: 10 ml/kg |
| | | | | body weight |
| Lithocholic acid | na | 434-13-9 | 1000 mg/kg body | in corn oil; administra- |
| | | | weight by gavage | tion volume: 5 ml/kg |
| | | | | bw |
| Methimazole | an | 60-56-0 | 100 mg/kg body weight | in drinking water, ad- |
| | | | by gavage | ministration volume: |
| | | | | 10 ml/kg bw |
| Oxaliplatin | na | 61825-94-3 | 2 mg/kg body weight | in 0.9% NaCl; admin- |
| | | | i.p., twice a week | istration volume: 3 |
| | | | | ml/kg bw |
| Probenecid | na | 57-66-9 | 800 mg/kg body weight | in corn oil, administra- |
| | | | by gavage | tion volume: 5 ml/kg |
| | | | | bw |
| Triethanolamine | Tri-(2-hydroxyethyl) amine | 102-71-6 | 2,000 mg/kg body | in aqua bidest., ad- |
| | | | weight by gavage | ministration volume: |
| | | | | 10 ml/kg body weight |
| Carboplatin | cis-Diammine(1,1- | 41575-94-4 | 2 mg/kg body weight | In 0.9% NaCl; admin- |
| | cyclobutanedicarboxylato) platinum | | i.p., twice a week | istration volume: 3 |
| | | | | ml/kg bw |
| Cisplatin | na | 15663-27-1 | 0.1 mg/kg body weight | in 0.9% NaCl; admin- |
| | | | i.p. | istration volume: 3 |
| | | | | ml/kg bw; twice a |
| | | | | week. |
| Ibuprofen | na | 15687-27-1 | 30 mg/kg bw by gavage | in drinking water con- |
| | | | | taining 0.5% CMC |
| | | | | (Tylose CB30000), |
| | | | | administration volume: |
| | | | | 10 ml/kg bw |
| Oxaliplatin *) | na | 61825-94-3 | 0.7 mg/kg body weight | in 0.9% NaCl; admin- |
| | | | i.p., twice a week | istration volume: 3 |
| | | | | ml/kg bw |
| Cyclophosphamide monohydrate | 1-Bromonaphthalene | 6055-19-2 | 8 mg/kg body weight | in drinking water, ad- |
| | | | by gavage (days 0 to | ministration volume: |
| | | | 9), 4 mg/kg body | 10 ml/kg bw |
| | | | weight by gavage | |
| | | | (from day 10 on) | |
| Adriamycin hydrochloride | Doxorubicin | 25316-40-9 | 2 mg/kg bw subcuta- | in 0.9% NaCl; admin- |
| | | | neous, once weekly | istration volume: 1 |
| | | | | ml/kg bw; once weekly |
| | | | | on days 0, 6, 13, 20 |
| | | | | and 27 |
| Cytarabin | beta-D-arabinofuranoside | 147-94-4 | 100 mg/kg body weight | In 0.9% NaCl; admin- |
| | | | i.p. | istration volume 3 |
| | | | | ml/kg body weight |

*) low dose for bone marrow supression only

TABLE 11
Chemical/physical properties of selected analytes. These biomarkers are characterized herein by chemical and physical properties.

| Metabolite | Fragmentation pattern (GC-MS) and description |
|---|---|
| 3-O-Methylsphingosine (d18:1) | 3-O-Methylsphingosine (d18:1) exhibits the following characteristic ionic fragments when |
| | detected with GC/MS, applying electron impact (EI) ionization mass spectrometry, after |
| | acidic methanolysis and derivatisation with 2% O-methylhydroxylamine-hydrochlorid in |
| | pyridine and subsequently with N-methyl-N-trimethylsilyltrifluoracetamid: MS (EI, 70 eV): |
| | m/z (%): 204 (100), 73 (18), 205 (16), 206 (7), 354 (4), 442 (1). |
| 5-O-Methylsphingosine (d18:1) | 5-O-Methylsphingosine (d18:1) exhibits the following characteristic ionic fragments when |
| | detected with GC/MS, applying electron impact (EI) ionization mass spectrometry, after |
| | acidic methanolysis and derivatisation with 2% O-methylhydroxylamine-hydrochlorid in |
| | pyridine and subsequently with N-methyl-N-trimethylsilyltrifluoracetamid: MS (EI, 70 eV): |
| | m/z (%): 250 (100), 73 (34), 251 (19), 354 (14), 355 (4), 442 (1). |
| Cholesterolester No 01 | Metabolite belongs to the class of cholesterolesters. It exhibits the following characteristic |
| | ionic species when detected with LC/MS, applying electro-spray ionization (ESI) mass |
| | spectrometry: mass-to-charge ratio (m/z) of the positively charged ionic species is 369.2 |
| | (+/−0.5). |
| Choline plasmalogen No 01 | Metabolite belongs to the class of choline plasmalogens. It exhibits the following charac- |
| | teristic ionic species when detected with LC/MS, applying electro-spray ionization (ESI) |
| | mass spectrometry: mass-to-charge ratio (m/z) of the positively charged ionic species is |
| | 772.6 (+/−0.5). |
| Choline plasmalogen No 02 | Metabolite belongs to the class of choline plasmalogens. It exhibits the following charac- |
| | teristic ionic species when detected with LC/MS, applying electro-spray ionization (ESI) |
| | mass spectrometry: mass-to-charge ratio (m/z) of the positively charged ionic species is |
| | 767 (+/−0.5). |
| Choline plasmalogen No 03 | Metabolite belongs to the class of choline plasmalogens. It exhibits the following charac- |
| | teristic ionic species when detected with LC/MS, applying electro-spray ionization (ESI) |
| | mass spectrometry: mass-to-charge ratio (m/z) of the positively charged ionic species is |
| | 768.8 (+/−0.5). |
| DAG (C18:1, C18:2) | DAG (C18:1, C18:2) represents the sum parameter of diacylglycerols containing the com- |
| | bination of a C18:1 fatty acid unit and a C18:2 fatty acid unit. The mass-to-charge ratio |
| | (m/z) of the ionised species is 641.6 Da (+/−0.5 Da). |
| Eicosaenoic acid (C20:1) No | Eicosaenoic acid (C20:1) exhibits the following characteristic ionic fragments when de- |
| 02 | tected with GC/MS, applying electron impact (EI) ionization mass spectrometry, after |
| | acidic methanolysis and derivatisation with 2% O-methylhydroxylamine-hydrochlorid in |
| | pyridine and subsequently with N-methyl-N-trimethylsilyltrifluoracetamid: MS (EI, 70 eV): |
| | m/z (%): 55 (100), 69 (75), 41 (57), 83 (54), 74 (53), 97 (45), 110 (20), 292 (13), 293 (13), |
| | 124 (12), 250 (9), 152 (8), 138 (8), 208 (7), 324 (2). |
| Glycerol phosphate, lipid frac- | Glycerol phosphate, lipid fraction represents the sum parameter of metabolites containing |
| tion | a glycerol-2-phosphate or a glycerol-3-phosphate moiety and being present in the lipid |
| | fraction after extraction and separation of the extract into a polar and a lipid fraction. |
| Lysophosphatidylcholine | Lysophosphatidylcholine (C17:0) represents the sum parameter of lysoglycerophosphorylcholines |
| (C17:0) | containing a C17:0 fatty acid unit. If detected with LC/MS, applying electro-spray |
| | ionization (ESI) mass spectrometry, the mass-to-charge ratio (m/z) of the positively |
| | charged ionic species is 510.4 Da (+/−0.5 Da). |
| Lysophosphatidylcholine | Lysophosphatidylcholine (C18:0) represents the sum parameter of lysoglycerophosphorylcholines |
| (C18:0) | containing a C18:0 fatty acid unit. If detected with LC/MS, applying electro- |
| | spray ionization (ESI) mass spectrometry, the mass-to-charge ratio (m/z) of the positively |
| | charged ionic species is 546.6 Da (+/−0.5 Da). |
| Lysophosphatidylcholine | Lysophosphatidylcholine (C18:1) represents the sum parameter of lysoglycerophosphorylcholines |
| (C18:1) | containing a C18:1 fatty acid unit. If detected with LC/MS, applying electro- |
| | spray ionization (ESI) mass spectrometry, the mass-to-charge ratio (m/z) of the positively |
| | charged ionic species is 522.2 Da (+/−0.5 Da). |
| Lysophosphatidylcholine | Lysophosphatidylcholine (C18:2) represents the sum parameter of lysoglycerophosphorylcholines |
| (C18:2) | containing a C18:2 fatty acid unit. If detected with LC/MS, applying |
| | electro-spray ionization (ESI) mass spectrometry, the mass-to-charge ratio (m/z) of the |
| | positively charged ionic species is 542.4 Da (+/−0.5 Da). |
| Lysophosphatidylcholine | Lysophosphatidylcholine (C20:4) represents the sum parameter of lysoglycerophosphorylcholines |
| (C20:4) | containing a C20:4 fatty acid unit. If detected with LC/MS, applying electro- |
| | spray ionization (ESI) mass spectrometry, the mass-to-charge ratio (m/z) of the positively |
| | charged ionic species is 544.4 Da (+/−0.5 Da). |
| Lysophosphatidylethanolamine | Lysophosphatidylethanolamine (C22:5) exhibits the following characteristic ionic species |
| (C22:5) | when detected with LC/MS, applying electro-spray ionization (ESI) mass spectrometry: |
| | mass-to-charge ratio (m/z) of the positively charged ionic species is 528.2 (+/−0.5). |
| Phosphatidylcholine | Phosphatidylcholine (C16:0/C16:0) represents the sum parameter of glycerophosphorylcholines |
| (C16:0, C16:0) | containing either the combination of of two C16:0 fatty acid units. The mass-to- |
| | charge ratio (m/z) of the ionised species is 734.8 Da (+/−0.5 Da). |
| Phosphatidylcholine | Phosphatidylcholine (C16:0, C20:5) exhibits the following characteristic ionic species |
| (C16:0, C20:5) | when detected with LC/MS, applying electro-spray ionization (ESI) mass spectrometry: |
| | mass-to-charge ratio (m/z) of the positively charged ionic species is 780.8 (+/−0.5). |
| Phosphatidylcholine | Phosphatidylcholine (C16:1, C18:2) represents the sum parameter of glycerophosphorylcholines |
| (C16:1, C18:2) | containing the combination of a C16:1 fatty acid unit and a C18:2 |
| | fatty acid unit. If detected with LC/MS, applying electro-spray ionization (ESI) mass spec- |
| | trometry, the mass-to-charge ratio (m/z) of the positively charged ionic species is 756.8 |
| | Da (+/−0.5 Da). |
| Phosphatidylcholine | Phosphatidylcholine (C18:0, C18:1) represents the sum parameter of glycerophosphorylcholines |
| (C18:0, C18:1) | containing the combination of a C18:0 fatty acid unit and a C18:1 fatty acid unit. |
| | If detected with LC/MS, applying electro-spray ionization (ESI) mass spectrometry, the |
| | mass-to-charge ratio (m/z) of the positively charged ionic species is 788.6 Da (+/−0.5 |
| | Da). |
| Phosphatidylcholine | Phosphatidylcholine (C18:0, C18:2) represents the sum parameter of glycerophosphorylcholines |
| (C18:0, C18:2) | containing the combination of a C18:0 fatty acid unit and a C18:2 fatty acid unit. |
| | If detected with LC/MS, applying electro-spray ionization (ESI) mass spectrometry, the |
| | mass-to-charge ratio (m/z) of the positively charged ionic species is 786.6 Da (+/−0.5 |
| | Da). |
| Phosphatidylcholine | Phosphatidylcholine (C18:0, C20:3) exhibits the following characteristic ionic species |
| (C18:0, C20:3) | when detected with LC/MS, applying electro-spray ionization (ESI) mass spectrometry: |
| | mass-to-charge ratio (m/z) of the positively charged ionic species is 812.6 (+/−0.5). |
| Phosphatidylcholine | Phosphatidylcholine (C18:0, C20:4) represents the sum parameter of glycerophosphorylcholines |
| (C18:0, C20:4) | containing the combination of a C18:0 fatty acid unit and a C20:4 fatty acid unit. |
| | If detected with LC/MS, applying electro-spray ionization (ESI) mass spectrometry, the |
| | mass-to-charge ratio (m/z) of the positively charged ionic species is 810.8 Da (+/−0.5 |
| | Da). |
| Phosphatidylcholine | Phosphatidylcholine (C18:0, C22:6) represents the sum parameter of glycerophosphorylcholines |
| (C18:0, C22:6) | containing the combination of a C18:0 fatty acid unit and a C22:6 fatty acid unit. |
| | If detected with LC/MS, applying electro-spray ionization (ESI) mass spectrometry, the |
| | mass-to-charge ratio (m/z) of the positively charged ionic species is 834.8 Da (+/−0.5 |
| | Da). |
| Phosphatidylcholine | Phosphatidylcholine (C16:0/C20:3 C18:1/C18:2) represents the sum parameter of glycerophosphorylcholines |
| (C18:1, C18:2) | containing the combination of a C18:1 fatty acid unit and a C18:2 |
| | fatty acid unit. The mass-to-charge ratio (m/z) of the ionised species is 784.6 Da (+/−0.5 |
| | Da). |
| Phosphatidylcholine | Phosphatidylcholine (C16:0/C22:6 C18:2/C20:4) represents the sum parameter of glycerophosphorylcholines |
| (C18:2, C20:4) | containing either the combination of a C16:0 fatty acid unit and a |
| | C22:6 fatty acid unit or the combination of a C18:2 fatty acid unit and a C20:4 fatty acid |
| | unit. The mass-to-charge ratio (m/z) of the ionised species is 806.6 Da (+/−0.5 Da). |
| Phosphatidylcholine No 02 | Metabolite belongs to the class of glycerophosphocholines. It exhibits the following char- |
| | acteristic ionic species when detected with LC/MS, applying electro-spray ionization (ESI) |
| | mass spectrometry: mass-to-charge ratio (m/z) of the positively charged ionic species is |
| | 808.4 (+/−0.5). |
| Phosphatidylcholine No 04 | Metabolite belongs to the class of glycerophosphocholines. It exhibits the following char- |
| | acteristic ionic species when detected with LC/MS, applying electro-spray ionization (ESI) |
| | mass spectrometry: mass-to-charge ratio (m/z) of the positively charged ionic species is |
| | 796.8 (+/−0.5). |
| Sphingomyelin (d18:1, C23:0) | Sphingomyelin (d18:1, C23:0) exhibits the following characteristic ionic species when de- |
| | tected with LC/MS, applying electro-spray ionization (ESI) mass spectrometry: mass-to- |
| | charge ratio (m/z) of the positively charged ionic species is 801.8 (+/−0.5). |
| Sphingomyelin (d18:1, C24:0) | Sphingomyelin (d18:1, C24:0) represents the sum parameter of sphingomyelins contain- |
| | ing the combination of a d18:1 long-chain base unit and a C24:0 fatty acid unit. If detect- |
| | ed with LC/MS, applying electro-spray ionization (ESI) mass spectrometry, the mass-to- |
| | charge ratio (m/z) of the positively charged ionic species is 815.8 Da (+/−0.5 Da). |
| Sphingomyelin (d18:2, C16:0) | Sphingomyelin (d18:2, C16:0) exhibits the following characteristic ionic species when de- |
| | tected with LC/MS, applying electro-spray ionization (ESI) mass spectrometry: mass-to- |
| | charge ratio (m/z) of the positively charged ionic species is 723.6 (+/−0.5). |
| Sphingomyelin (d18:2, C18:0) | Sphingomyelin (d18:2, C18:0) exhibits the following characteristic ionic species when de- |
| | tected with LC/MS, applying electro-spray ionization (ESI) mass spectrometry: mass-to- |
| | charge ratio (m/z) of the positively charged ionic species is 729.8 (+/−0.5). |
| TAG (C16:0, C16:1) | Metabolite represents the sum of triacylglycerides containing the combination of a C16:0 |
| | fatty acid unit and a C16:1 fatty acid unit. It exhibits the following characteristic ionic spe- |
| | cies when detected with LC/MS, applying electro-spray ionization (ESI) mass spectrome- |
| | try: mass-to-charge ratio (m/z) of the positively charged ionic species is 549.6 (+/−0.5). |
| TAG (C16:0, C18:1, C18:3) | TAG (C16:0, C18:1, C18:3) exhibits the following characteristic ionic species when detect- |
| | ed with LC/MS, applying electro-spray ionization (ESI) mass spectrometry: mass-to- |
| | charge ratio (m/z) of the positively charged ionic species is 855.6 (+/−0.5). |
| TAG (C16:0, C18:2) | Metabolite represents the sum of triacylglycerides containing the combination of a C16:0 |
| | fatty acid unit and a C18:2 fatty acid unit. It exhibits the following characteristic ionic spe- |
| | cies when detected with LC/MS, applying electro-spray ionization (ESI) mass spectrome- |
| | try: mass-to-charge ratio (m/z) of the positively charged ionic species is 575.6 (+/−0.5). |
| TAG (C18:1, C18:2) | Metabolite represents the sum of triacylglycerides containing the combination of a C18:1 |
| | fatty acid unit and a C18:2 fatty acid unit. It exhibits the following characteristic ionic spe- |
| | cies when detected with LC/MS, applying electro-spray ionization (ESI) mass spectrome- |
| | try: mass-to-charge ratio (m/z) of the positively charged ionic species is 601.6 (+/−0.5). |
| TAG (C18:2, C18:2) | Metabolite represents the sum of triacylglycerides containing the combination of a C18:2 |
| | fatty acid unit and a C18:2 fatty acid unit. It exhibits the following characteristic ionic spe- |
| | cies when detected with LC/MS, applying electro-spray ionization (ESI) mass spectrome- |
| | try: mass-to-charge ratio (m/z) of the positively charged ionic species is 599.6 (+/−0.5). |
| TAG (C18:2, C18:3) | Metabolite represents the sum of triacylglycerides containing the combination of a C18:2 |
| | fatty acid unit and a C18:3 fatty acid unit. It exhibits the following characteristic ionic spe- |
| | cies when detected with LC/MS, applying electro-spray ionization (ESI) mass spectrome- |
| | try: mass-to-charge ratio (m/z) of the positively charged ionic species is 597.6 (+/−0.5). |
| TAG (DAG-Fragment) | Metabolite belongs to the class of triacylglycerides. It exhibits the following characteristic |
| | ionic species when detected with LC/MS, applying electro-spray ionization (ESI) mass |
| | spectrometry: mass-to-charge ratio (m/z) of the positively charged ionic species is 600.6 |
| | (+/−0.5). |
| TAG No 01 | Metabolite belongs to the class of triacylglycerides. It exhibits the following characteristic |
| | ionic species when detected with LC/MS, applying electro-spray ionization (ESI) mass |
| | spectrometry: mass-to-charge ratio (m/z) of the positively charged ionic species is 547.6 |
| | (+/−0.5). |
| TAG No 02 | Metabolite belongs to the class of triacylglycerides. It exhibits the following characteristic |
| | ionic species when detected with LC/MS, applying electro-spray ionization (ESI) mass |
| | spectrometry: mass-to-charge ratio (m/z) of the positively charged ionic species is 695.6 |
| | (+/−0.5). |
| TAG No 05 | Metabolite belongs to the class of triacylglycerides. It exhibits the following characteristic |
| | ionic species when detected with LC/MS, applying electro-spray ionization (ESI) mass |
| | spectrometry: mass-to-charge ratio (m/z) of the positively charged ionic species is 879.6 |
| | (+/−0.5). |
| TAG No 059 | Metabolite belongs to the class of triacylglycerides. It exhibits the following characteristic |
| | ionic species when detected with LC/MS, applying electro-spray ionization (ESI) mass |
| | spectrometry: mass-to-charge ratio (m/z) of the positively charged ionic species is 904 |
| | (+/−0.5). |
| TAG No 07 | Metabolite belongs to the class of triacylglycerides. It exhibits the following characteristic |
| | ionic species when detected with LC/MS, applying electro-spray ionization (ESI) mass |
| | spectrometry: mass-to-charge ratio (m/z) of the positively charged ionic species is 853.6 |
| | (+/−0.5). |

TABLE 12a
Markers for hematotoxicity (blood anemia) in male rats; All metabolites
showed significant down-regulation changes (p-Value ≦ 0.2).

| | 4-Chloro-3- | | |
|---|---|---|---|
| | nitroaniline | Aniline | 2-Chloroaniline |
| Metabolite | m7 | m7 | m7 |
| Ketoleucine | 0.61 | 0.94 | 0.60 |
| 3,4-Dihydroxyphenylglycol | 0.53 | 0.84 | 0.87 |
| (DOPEG) | | | |
| 3,4-Dihydroxyphenylalanine | 0.93 | 0.81 | 0.76 |
| (DOPA) | | | |

TABLE 12b
Markers for hematotoxicity (blood anemia) in male rats; All metabolites
showed significant up-regulation changes (p-Value ≦ 0.2).

| | 4-Chloro-3 | | |
|---|---|---|---|
| | nitroaniline | Aniline | 2-Chloroaniline |
| Metabolite | m7 | m7 | m7 |
| Ribal | 1.89 | 1.66 | 1.24 |
| Cytosine | 1.75 | 1.62 | 1.41 |
| 3-Hydroxyindole | 1.25 | 1.61 | 1.40 |
| 3-Indoxylsulfate | 1.34 | 1.64 | 1.68 |
| Serotonin (5-HT) | 9.50 | NA | 2.64 |
| 3-Methoxytyrosine | 1.90 | 1.35 | 1.48 |

1. A method for diagnosing hematopoietic toxicity comprising:
(a) determining the amount of at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9, 12a or 12b in a test sample of a subject suspected to suffer from hematopoietic toxicity, and
(b) comparing the amounts determined in step (a) to a reference, whereby hematopoietic toxicity is to be diagnosed.

2. The method of claim 1, wherein said subject has been brought into contact with a compound suspected to be capable of inducing hematopoietic toxicity.

3. A method of determining whether a compound is capable of inducing hematopoietic toxicity in a subject comprising:
(a) determining the amount of at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9, 12a or 12b in a sample of a subject which has been brought into contact with a compound suspected to be capable of inducing hematopoietic toxicity and
(b) comparing the amounts determined in step (a) to a reference, whereby the capability of the compound to induce hematopoietic toxicity is determined.

4. The method of claim 2, wherein said compound is at least one compound selected from the group consisting of: 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen.

5. The method of claim 1, wherein said reference is derived from (i) a subject or group of subjects which suffers from hematopoietic toxicity or (ii) a subject or group of subjects which has been brought into contact with at least one compound selected from the group consisting of: 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen.

6. The method of claim 5, wherein essentially identical amounts for the biomarkers in the test sample and the reference are indicative for hematopoietic toxicity.

7. The method of claim 1, wherein said reference is derived from (i) a subject or group of subjects known to not suffer from hematopoietic toxicity or (ii) a subject or group of subjects which has not been brought into contact with at least one compound selected from the group consisting of: 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen.

8. The method of claim 1, wherein said reference is a calculated reference for the biomarkers for a population of subjects.

9. The method of claim 7, wherein amounts for the biomarkers which differ in the test sample in comparison to the reference are indicative for hematopoietic toxicity.

10. A method of identifying a substance for treating hematopoietic toxicity comprising the steps of:
(a) determining in a sample of a subject suffering from hematopoietic toxicity which has been brought into contact with a candidate substance suspected to be capable of treating hematopoietic toxicity the amount of at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9, 12a or 12b; and
(b) comparing the amounts determined in step (a) to a reference, whereby a substance capable of treating hematopoietic toxicity is to be identified.

11. The method of claim 10, wherein said reference is derived from (i) a subject or group of subjects which suffers from hematopoietic toxicity or (ii) a subject or group of subjects which has been brought into contact with at least one compound selected from the group consisting of: 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen.

12. The method of claim 11, wherein amounts for the biomarkers which differ in the test sample and the reference are indicative for a substance capable of treating hematopoietic toxicity.

13. The method of claim 10, wherein said reference is derived from (i) a subject or group of subjects known to not suffer from hematopoietic toxicity or (ii) a subject or group of subjects which has not been brought into contact with at least one compound selected from the group consisting of: 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen.

14. The method of claim 10, wherein said reference is a calculated reference for the biomarkers in a population of subjects.

15. The method of claim 13, wherein essentially identical amounts for the biomarkers in the test sample and the reference are indicative for a substance capable of treating hematopoietic toxicity.

16. (canceled)

17. A device for diagnosing hematopoietic toxicity in a sample of a subject suspected to suffer therefrom comprising:
(a) an analyzing unit comprising a detection agent for at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9, 12a or 12b which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby hematopoietic toxicity is diagnosed.

18. The device of claim 17, wherein said stored reference is a reference derived from a subject or a group of subjects known to suffer from hematopoietic toxicity or a subject or group of subjects which has been brought into contact with at least one compound selected from the group consisting of 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen and said data processor executes instructions for comparing the amount of the at least one biomarker determined by the analyzing unit to the stored reference, wherein an essentially identical amount of the at least one biomarker in the test sample in comparison to the reference is indicative for the presence of hematopoietic toxicity or wherein an amount of the at least one biomarker in the test sample which differs in comparison to the reference is indicative for the absence of hematopoietic toxicity.

19. The device of claim 17, wherein said stored reference is a reference derived from a subject or a group of subjects known to not suffer from hematopoietic toxicity or a subject or group of subjects which has not been brought into contact with at least one compound selected from the group consisting of 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen and said data processor executes instructions for comparing the amount of the at least one biomarker determined by the analyzing unit to the stored reference, wherein an amount of the at least one biomarker in the test sample which differs in comparison to the reference is indicative for the presence of hematopoietic toxicity or wherein an essentially identical amount of the at least one biomarker in the test sample in comparison to the reference is indicative for the absence of hematopoietic toxicity.

20. A kit for diagnosing hematopoietic toxicity comprising a detection agent for the at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9, 12a or 12b and standards for the at least one biomarker the concentration of which is derived from (i) a subject or a group of subjects known to suffer from hematopoietic toxicity or a subject or group of subjects which has been brought into contact with at least one compound selected from the group consisting of 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen or derived (ii) from a subject or a group of subjects known to not suffer from hematopoietic toxicity or a subject or group of subjects which has not been brought into contact with at least one compound selected from the group consisting of 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen.

21. The method of claim 3, wherein said compound is at least one compound selected from the group consisting of: 1,3-Dinitrobenzene, 1,4-Dinitrobenzene, 2-Butoxyethanol, 2-Chloroaniline, Cyclohexanone oxime (CHO), 4-Chloro-3-nitroaniline, Adriamycin hydrochloride, Aniline, Cyclosporin A, Epoxiconazole, Flutamide, Lead acetate trihydrate, Linuron, Lithocholic acid, Methimazole, Methylprednisolone, Oxaliplatin, Probenecid, Tacrolimus, Triethanolamine, Carboplatin, Cisplatin, Cyclophosphamide monohydrate, Cytarabin, and Ibuprofen.
